(19) United States
(12) Patent Application Publication
(10) Pub. No.: US 2002/0164576 A1
(43) Pub. Date: Nov. 7, 2002

(54) Methods for diagnosis and treatment of diseases associated with altered expression of Nrf2

(76) Inventors: Finn Skou Pedersen, Aarhus V (DK); Annette Balle Soerensen, Aarhus N (DK); Helle Moving, Aarhus V (DK)

Correspondence Address: ALBRITTON & HERBERT LLP, FLEHR HOHBACH TEST, Suite 3400, Four Embarcadero Center, San Francisco, CA, 94111-4187, US

(21) Appl. No.: 09/962,855

(22) Filed: Sep. 24, 2001

Related U.S. Application Data
(63) Continuation of application No. 09/668,644, filed on Sep. 22, 2000.

(51) Int. Cl. C12Q 1/00; C12Q 1/68; C07H 21/04; C12N 9/00; C12N 5/06

(52) U.S. Cl. 435/4; 435/6; 435/325; 435/320.1; 435/183; 536/23.2; 514/1

(57) ABSTRACT

The present invention relates to novel sequences for use in diagnosis and treatment of lymphoma and leukemia. In addition, the present invention describes the use of novel compositions for use in screening methods.

This application is a continuing application of U.S. Ser. No. 09/668,644 filed Sep. 22, 2000, which is expressly incorporated herein by reference.

### FIELD OF THE INVENTION

The present invention relates to novel sequences for use in diagnosis and treatment of lymphoma and leukemia, as well as the use of the novel compositions in screening methods.

### BACKGROUND OF THE INVENTION

Lymphomas are a collection of cancers involving the lymphatic system and are generally categorized as Hodgkin's disease and Non-Hodgkin lymphoma. Hodgkin's lymphomas are of B lymphocyte origin. Non-Hodgkin lymphomas are a collection of over 30 different types of cancers including T and B lymphomas. Leukemias are cancerous diseases of the blood forming tissues and include B and T cell lymphocytic leukemias. Leukemias are characterized by an abnormal and persistent increase in the number of leukocytes and the amount of bone marrow, with enlargement of the spleen and lymph nodes.

Oncogenes are genes that can cause cancer. Cancer can occur by a wide variety of mechanisms, including infection of cells by viruses containing oncogenes, activation of protooncogenes in the host genome, and mutations of protooncogenes and tumor suppressor genes.

There are a number of viruses known to be involved in human cancer as well as cancer in lower animals. Of particular interest here are viruses that do not contain oncogenes themselves; these are slow-transforming retroviruses. They induce tumors by integrating into the host genome and affecting neighboring protooncogenes or tumor suppressor genes in a variety of ways and through a variety of mechanisms including promoter insertion, enhancer insertion, and/or gene truncation. The analysis of sequences at or near viral DNA insertion sites has led to the identification of a number of new protooncongenes and tumor suppressor genes that when modified or modulated give rise to cancer in cells of the hematopoietic system.

With respect to lymphoma and leukemia, murine leukemia retrovirus (MuLV), such as SL3-3 or Akv, is a potent inducer of tumors when inoculated into susceptible newborn mice, or when carried in the germline. A number of viral sequences have been identified as relevant in the induction of lymphoma and leukemia by analyzing the insertion sites; see Sorensen et al., J. of Virology 74:2161 (2000); Hansen et al., Genome Res. 10(2):237-43 (2000); Sorensen et al., J. Virology 70:4063 (1996); Sorensen et al., J. Virology 67:7118 (1993); Joosten et al., Virology 268:308 (2000); and Li et al., Nature Genetics 23:348 (1999); all of which are expressly incorporated by reference herein.

Accordingly, it is an object of the invention to provide mammalian gene sequences involved in oncogenesis, particularly with respect to cells of the hematopoietic system.

In this regard, the present invention provides a mammalian Nrf2 gene which is shown herein to be involved in lymphoma.

The Nrf2 gene encodes a DNA binding transcriptional regulatory protein (transcription factor) belonging to the “cap 'n collar” subfamily of the basic leucine zipper family of transcription factors (Chan et al., PNAS 93:13943-13948, 1996; Moi et al., PNAS 91:9926-9930, 1994). The Nrf2 gene produces a 2.2 kb transcript which predicts a 66 kDa protein (Moi et al., PNAS 91:9926-9930, 1994). The Nrf2 protein binds to a DNAse hypersensitive site located in the β-globin locus control region (Moi et al., PNAS 91:9926-9930, 1994), as well as to the antioxidant response element (ARE) which is found in the regulatory regions of many detoxifying enzyme genes (Venugopal et al., Oncogene, 17:3145-3156, 1998).

Nrf2 gene function is not required for normal development, as evidenced by homozygous disruption of the Nrf2 loci in transgenic mice (Chan et al., PNAS 93:13943-13948, 1996). However, loss of Nrf2 gene function compromises the ability of haematopioetic cells to endure oxidative stress (Ishii et al., J. Biol. Chem., 275:16023-16029, 2000; Enomoto et al., Toxicol. Sci., 59:169-177, 2001) and sensitizes cells to the carcinogenic activity of oxidative agents (Ramos-Gomez et al., PNAS, 98:3410-3415, 2001).

Nrf2 proteins are capable of interacting with other transcription factors, including Jun proteins (Venugopal et al., Oncogene, 17:3145-3156, 1998) and Maf proteins (Marini et al., J. Biol. Chem., 272-16490-16497, 1997). Jun proteins appear to cooperate with Nrf2 to regulate the transcription of target genes (Venugopal et al., Oncogene, 17:3145-3156, 1998) while Maf proteins appear to antagonize the transcription promoting activity of Nrf2 protein (Nguyen et al., J. Biol. Chem., 275:15466-15473, 2000). In addition, the human cytomegalovirus protein IE-2 has also been found to interact with Nrf2 and to inhibit its transcription promoting activity (Huang et al., J. Biol. Chem., 275:12313-12320, 2000).

Despite being dispensable for the normal development of lymphoid cells and tissues, which includes the normal processes of B cell and T cell determination, differentiation, proliferation, and death, it is demonstrated herein that dysregulation of the Nrf2 gene leads to lymphoma.

### SUMMARY OF THE INVENTION

In accordance with the objects outlined above, the present invention provides methods for screening for compositions which modulate lymphomas. Also provided herein are methods of inhibiting proliferation of a cell, preferably a lymphoma cell. Methods for the diagnosis of lymphomas are also provided herein. Compositions for the therapeutic treatment of lymphomas, as well as compositions for prophylaxis against lymphomas, are also provided.

In one aspect the present invention provides nucleic acid sequences associated with lymphoma and referred to herein as lymphoma associated (LA) genes. In one aspect, the present invention provides amino acid sequences associated with lymphoma and referred to herein as lymphoma associated proteins (LAPs). The products of LA genes include LA proteins.

In one aspect the present invention provides an LA protein known as Nrf2. In a preferred embodiment Nrf2 comprises the amino acid sequence set forth in SEQ ID NO:2 and at Genbank Accession number AAA68291, which is encoded by the Nrf2 nucleic acid sequence set forth by nucleotides 298 to 2043 in SEQ ID NO:1 and at Genbank Accession Number U20532. In one aspect the present invention provides an LA nucleic acid referred to herein as Nrf2. In a preferred embodiment the Nrf2 nucleic acid comprises the nucleic acid sequence set forth in SEQ ID NO:1 and at Genbank Accession number U20532, which encodes an Nrf2 protein.

In one aspect the present invention provides an LA protein known as Nrf2 comprising the amino acid sequence set forth in SEQ ID NO:4 and at Genbank Accession number NP_{—}006155, which is encoded by the Nrf2 nucleic acid sequence set forth by nucleotides 40 to 1809 in SEQ ID NO:3 and at Genbank Accession Number NM_{—}006164. In one aspect the present invention provides an LA nucleic acid referred to herein as Nrf2 and comprising the nucleic acid sequence set forth in SEQ ID NO:3 and at Genbank Accession number NM_{—}006164, which encodes an Nrf2 protein.

Also provided herein are Nrf2 nucleic acids comprising a nucleic acid sequence having at least about 90% identity to the nucleic acid sequence set forth in SEQ ID NO:1 and at Genbank Accession number U20532, or complements thereof.

Also provided herein are Nrf2 nucleic acids comprising a nucleic acid sequence having at least about 90% identity to the nucleic acid sequence set forth in SEQ ID NO:3 and at Genbank accession number NM_{—}006164, or complements thereof.

Also provided herein are Nrf2 nucleic acids which will hybridize under high stringency conditions to a nucleic acid comprising the nucleic acid sequence set forth in SEQ ID NO:1 and at Genbank accession number U20532, or complements thereof.

Also provided herein are Nrf2 nucleic acids which will hybridize under high stringency conditions to a nucleic acid comprising the nucleic acid sequence set forth in SEQ ID NO:3 and at Genbank accession number NM_{—}006164, or complements thereof.

Also provided herein are Nrf2 proteins encoded by Nrf2 nucleic acids as described herein.

Also provided herein are Nrf2 proteins comprising an amino acid sequence having at least about 90% identity to the amino acid sequence set forth in SEQ ID NO:2 and at Genbank accession number AAA68291.

Also provided herein are Nrf2 proteins comprising an amino acid sequence having at least about 90% identity to the amino acid sequence set forth in SEQ ID NO:4 and at Genbank accession number NP_{—}006155.

Also provided herein are Nrf2 genes encoding Nrf2 proteins comprising an amino acid sequence having at least about 90% identity to the amino acid sequence set forth in SEQ ID NO:2 and at Genbank bank accession number AAA68291.

Also provided herein are Nrf2 genes encoding Nrf2 proteins comprising an amino acid sequence having at least about 90% identity to the amino acid sequence set forth in SEQ ID NO:4 and at Genbank accession number NP_{—}006155.

In one aspect, the present invention provides a method for screening for a candidate bioactive agent capable of modulating the activity of an Nrf2 gene. In one embodiment, such a method comprises adding a candidate agent to a cell and determining the level of expression of an Nrf2 gene in the presence and absence of the candidate agent. In a preferred embodiment, an Nrf2 gene comprises the nucleic acid sequence set forth in SEQ ID NO:1 and at Genbank accession number U20532. In another preferred embodiment, an Nrf2 gene comprises the nucleic acid sequence set forth in SEQ ID NO:3 and at Genbank accession number NM_{—}006164.

Further provided herein is a method for screening for a candidate bioactive agent capable of modulating the activity of an Nrf2 protein encoded by an Nrf2 gene. In one embodiment, such a method comprises contacting an Nrf2 protein or a cell comprising an Nrf2 protein, and a candidate bioactive agent, and determining the effect on the activity of the Nrf2 protein in the presence and absence of the candidate agent. In another embodiment, such a method comprises contacting a cell comprising an Nrf2 protein, and a candidate bioactive agent, and determining the effect on the cell in the presence and absence of the candidate agent. In a preferred embodiment, an Nrf2 protein comprises the amino acid sequence set forth in SEQ ID NO:2 and at Genbank accession number AAA68291, or a fragment thereof. In another preferred embodiment, an Nrf2 protein comprises the amino acid sequence set forth in SEQ ID NO:4 and at Genbank accession number NP_{—}006155, or a fragment thereof. In a preferred embodiment, an Nrf2 protein comprises an amino acid sequence encoded by the nucleic acid sequence set forth in SEQ ID NO:1 and at Genbank accession number U20532, or a fragment thereof. In another preferred embodiment, an Nrf2 protein comprises an amino acid sequence encoded by the nucleic acid sequence set forth in SEQ ID NO:3 and at Genbank accession number NM_{—}006164, or a fragment thereof. In one embodiment, an Nrf2 protein is a recombinant protein. In one embodiment, an Nrf2 protein is isolated. In one embodiment, an Nrf2 protein is cell-free, as in a cell lysate.

Also provided herein is a method for screening for a bioactive agent capable of binding to an Nrf2 protein encoded by an Nrf2 gene. In one embodiment, such a method comprises combining an Nrf2 protein or a cell comprising an Nrf2 protein, and a candidate bioactive agent, and determining the binding of the candidate agent to the Nrf2 protein. In a preferred embodiment, an Nrf2 protein comprises the amino acid sequence set forth in SEQ ID NO:2, or a fragment thereof. In another preferred embodiment, an Nrf2 protein comprises the amino acid sequence set forth in SEQ ID NO:4, or a fragment thereof. In a preferred embodiment, an Nrf2 protein comprises an amino acid sequence encoded by the nucleic acid sequence set forth in SEQ ID NO:1, or a fragment thereof. In another preferred embodiment, an Nrf2 protein comprises an amino acid sequence encoded by the nucleic acid sequence set forth in SEQ ID NO:3, or a fragment thereof. In one embodiment, an Nrf2 protein is a recombinant protein. In one embodiment, an Nrf2 protein is isolated. In one embodiment, an Nrf2 protein is cell-free, as in a cell lysate.

Also provided is a method for evaluating the effect of a candidate lymphoma drug, comprising administering the drug to a patient and removing a cell sample or a cell fraction sample from the patient. A gene expression profile for the sample is then determined, including determination of the expression of an Nrf2 gene. In a preferred embodiment, an Nrf2 gene comprises the nucleic acid sequence set forth in SEQ ID NO:1, or a fragment thereof. In another preferred embodiment, an Nrf2 gene comprises the nucleic acid sequence set forth in SEQ ID NO:3, or a fragment thereof. Such a method may further comprise comparing the expression profile of the patient sample to an expression profile of a healthy individual sample.

In a further aspect, a method for inhibiting the activity of an Nrf2 protein is provided. In one embodiment, the method comprises administering to a patient an inhibitor of an Nrf2 protein. In a preferred embodiment, an Nrf2 protein comprises the amino acid sequence set forth in SEQ ID NO:2 or a fragment thereof. In another preferred embodiment, an Nrf2 protein comprises the amino acid sequence set forth in SEQ ID NO:4 or a fragment thereof. In a preferred embodiment, an Nrf2 protein comprises an amino acid sequence encoded by the nucleic acid sequence set forth in SEQ ID NO:1 or a fragment thereof. In another preferred embodiment, an Nrf2 protein comprises an amino acid sequence encoded by the nucleic acid sequence set forth in SEQ ID NO:3 or a fragment thereof.

Also provided herein is a method for neutralizing Nrf2 protein activity with a bioactive agent. In a preferred embodiment, an Nrf2 protein comprises the amino acid sequence set forth in SEQ ID NO:2 or a fragment thereof. In another preferred embodiment, an Nrf2 protein comprises the amino acid sequence set forth in SEQ ID NO:4 or a fragment thereof. In a preferred embodiment, an Nrf2 protein comprises an amino acid sequence encoded by the nucleic acid sequence set forth in SEQ ID NO:1, or a fragment thereof. In another preferred embodiment, an Nrf2 protein comprises an amino acid sequence encoded by the nucleic acid sequence set forth in SEQ ID NO:3, or a fragment thereof. In one embodiment, such a method comprises contacting an Nrf2 protein with an agent that specifically modulates Nrf2 protein activity, in an amount sufficient to effect neutralization.

Moreover, provided herein is a biochip comprising a nucleic acid which encodes an Nrf2 protein or a portion thereof. In a preferred embodiment, an Nrf2 nucleic acid comprises the nucleic acid sequence set forth in SEQ ID NO:1, or complement thereof, or a fragment thereof or complement of a fragment thereof. In another preferred embodiment, an Nrf2 nucleic acid comprises the nucleic acid sequence set forth in SEQ ID NO:3, or complement thereof, or a fragment thereof or complement of a fragment thereof.

Also provided herein is a method for diagnosing or determining a predisposition for lymphomas, comprising sequencing at least one Nrf2 gene from an individual and determining the nucleic acid sequence of the Nrf2 gene or a fragment thereof. In a preferred embodiment, an Nrf2 gene comprises the nucleic acid sequence set forth in SEQ ID NO:1, or a fragment thereof. In another preferred embodiment, an Nrf2 gene comprises the nucleic acid sequence set forth in SEQ ID NO:3, or a fragment thereof.

Similarly provided are methods for determining lymphoma subtype and determining a prognosis for an individual having lymphoma, which comprise sequencing at least one Nrf2 gene from an individual and determining the nucleic acid sequence of the Nrf2 gene or a fragment thereof. In a preferred embodiment, an Nrf2 gene comprises the nucleic acid sequence set forth in SEQ ID NO:1, or a fragment thereof. In another preferred embodiment, an Nrf2 gene comprises the nucleic acid sequence set forth in SEQ ID NO:3, or a fragment thereof.

In yet another aspect of the invention, a method is provided for determining the number of copies of an Nrf2 gene in an individual. In a preferred embodiment, an Nrf2 gene comprises the nucleic acid sequence set forth in SEQ ID NO: 1, or complement thereof, or a fragment thereof or complement of a fragment thereof. In a preferred embodiment, an Nrf2 gene comprises the nucleic acid sequence set forth in SEQ ID NO:3, or complement thereof, or a fragment thereof or complement of a fragment thereof.

In yet another aspect of the invention, a method is provided for determining the chromosomal location of an Nrf2 gene. In a preferred embodiment, an Nrf2 gene comprises the nucleic acid sequence set forth in SEQ ID NO:1, or a fragment thereof. In another preferred embodiment, an Nrf2 gene comprises the nucleic acid sequence set forth in SEQ ID NO:3, or a fragment thereof. Such a method may be used to determine Nrf2 gene rearrangements or translocations. Without being bound by theory, Nrf2 gene rearrangement and translocation events appear to be important in the aetiology of lymphoma.

It is an object of this invention that the identification Nrf2 genes and recognition of their involvement in lymphoma provide diagnostic agents to distinguish between lymphoma subtypes, and analytical agents for further analysis of mechanisms involved in dysregulated growth and/or survival and/or apoptosis in cells of the hematopoietic system. An additional object of the invention is to provide appropriate and potentially novel targets for therapeutic interventions, particularly with regard to lymphoma, which are identified through the use of the diagnostic and analytical agents provided herein.

Without being bound by theory, it is recognized herein that the involvement of Nrf2 genes in the cellular dysregulation underlying lymphoma implicates genes having an Nrf2 DNA binding sequence in the cellular dysregulation underlying lymphoma. In a preferred embodiment, the Nrf2 DNA binding sequence is bound by an Nrf2 protein comprising the amino acid sequence set forth in SEQ ID NO:2 and at Genbank accession number AAA68291, or a fragment thereof. In another preferred embodiment, the Nrf2 DNA binding sequence is bound by an Nrf2 protein comprising the amino acid sequence set forth in SEQ ID NO:4 and at Genbank accession number NP_{—}006155, or a fragment thereof.

Novel sequences are also provided herein. Other aspects of the invention will become apparent to the skilled artisan by the following description of the invention.

### DETAILED DESCRIPTION OF THE INVENTION

The present invention is directed to sequences associated with lymphoma. The use of oncogenic retroviruses, whose sequences insert into the genome of the host organism resulting in lymphoma, allows the identification of host sequences involved in lymphoma. These sequences may then be used in a number of different ways, including diagnosis, prognosis, screening for modulators (including both agonists and antagonists), antibody generation (for immunotherapy and imaging), etc. These sequences also find use in the generation of animal models of human disease, as described below.

Accordingly, the present invention provides nucleic acid and protein sequences that are associated with lymphoma, herein termed “lymphoma/leukemia associated” or “lymphoma/leukemia defining” or “LA” sequences, or LA nucleic acids, or LA proteins.

In a preferred embodiment, the present invention sets forth LA nucleic acids referred to herein as Nrf2 nucleic acids. In another preferred embodiment, the present invention sets forth LA proteins referred to herein as Nrf2 proteins.

“Associated with lymphoma” means that the nucleotide or protein sequences are either differentially expressed or altered in lymphoma as compared to normal lymphoid cells and tissues. As outlined below, Nrf2 sequences may be up-regulated (i.e. expressed at a higher level) or down-regulated (i.e. expressed at a lower level) in lymphoma. Nrf2 sequences may also be altered (i.e., truncated sequences or sequences with a point mutation). Additionally, Nrf2 sequences may show either the same expression profile or an altered profile in lymphoma relative to normal lymphoid cells and tissues. In a preferred embodiment, the Nrf2 sequences are human sequences; however, as will be appreciated by those in the art, Nrf2 sequences from other organisms may be useful in animal models of disease and drug evaluation; thus, other Nrf2 sequences are provided, from vertebrates, including mammals, including rodents (rats, mice, hamsters, guinea pigs, etc.), primates, farm animals (including sheep, goats, pigs, cows, horses, etc). Nrf2 sequences from other organisms may be obtained using the techniques outlined below. Also appreciated by those in the art will be the use of human sequences in other animals and the use of sequences between different classes or species of animals generally.

Sequences can include both nucleic acid and amino acid sequences. In a preferred embodiment, the Nrf2 sequences are recombinant nucleic acids. By the term “recombinant nucleic acid” herein is meant nucleic acid, originally formed in vitro, in general, by the manipulation of nucleic acid by polymerases and endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of an Nrf2 protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

In a preferred embodiment, the Nrf2 sequences are nucleic acids. As will be appreciated by those in the art and is more fully outlined below, Nrf2 sequences are useful in a variety of applications, including diagnostic applications, which will detect naturally occurring nucleic acids, as well as screening applications; for example, biochips comprising nucleic acid probes to the Nrf2 sequences (or to Nrf2 mutations or variants) can be generated. In the broadest sense, then, by “nucleic acid” or “oligonucleotide” or grammatical equivalents herein means at least two nucleotides covalently linked together. A nucleic acid of the present invention will generally contain phosphodiester bonds, although in some cases, as outlined below (for example in antisense applications or when a candidate agent is a nucleic acid), nucleic acid analogs may be used that have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp169-176). Several nucleic acid analogs are described in Rawls, C & E News June 2, 1997 page 35. All of these references are hereby expressly incorporated by reference. These modifications of the ribose-phosphate backbone may be done for a variety of reasons, for example to increase the stability and half-life of such molecules in physiological environments or as probes on a biochip.

As will be appreciated by those in the art, all of these nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

Particularly preferred are peptide nucleic acids (PNA) including peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand (“Watson”) also defines the sequence of the other strand (“Crick”); thus the description of sequences herein also includes the complement sequences. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

An Nrf2 sequence can be initially identified by substantial nucleic acid and/or amino acid sequence homology to the Nrf2 sequences outlined herein. Such homology can be based upon the overall nucleic acid or amino acid sequence, and is generally determined as outlined below, using either homology programs or hybridization conditions.

The Nrf2 sequences of the invention were identified as follows; basically, infection of mice with murine leukemia viruses (MuLV; including SL3-3, Akv and mutants thereof) resulted in lymphoma. The Nrf2 sequence outlined herein comprises an insertion site for the virus and flanking endogenous sequences. In general, the retrovirus can cause lymphoma in three basic ways: first, by activating expression of a normally silent neighboring gene; second, by enhancing expression of a normally expressed neighboring gene; and third, by truncating a gene. By neighboring gene is meant a gene within 100 kb to 500 kb or more, more preferably 50 kb to 100 kb, more preferably 1 kb to 50 kb, of the insertion site. For example, retrovirus enhancers, including SL3-3, are known to act on genes up to approximately 200 kilobases of the insertion site.

In a preferred embodiment, Nrf2 sequences are those that are up-regulated in lymphoma; that is, the expression of these genes is higher in lymphoma as compared to normal lymphoid tissue of the same differentiation stage. “Up-regulation” as used herein means at least about 50%, more preferably at least about 100%, more preferably at least about 150%, more preferably, at least about 200%, with from 300 to at least 1000% being especially preferred. As is recognized by those in the art, upregulated gene expression may result from gene multiplication and/or translocation, though upregulation can occur by other means.

In a preferred embodiment, Nrf2 sequences are those that are down-regulated in lymphoma; that is, the expression of these genes is lower in lymphoma as compared to normal lymphoid tissue of the same differentiation stage. “Down-regulation” as used herein means at least about 50%, more preferably at least about 100%, more preferably at least about 150%, more preferably, at least about 200%, with from 300 to at least 1000% being especially preferred.

In a preferred embodiment, Nrf2 sequences are those that are altered but show either the same expression profile or an altered profile as compared to normal lymphoid tissue of the same differentiation stage. “Altered Nrf2 sequences” as used herein refers to sequences which are truncated, contain insertions, deletions, fusions, or contain point mutations.

In one embodiment, the present invention provides an Nrf2 gene comprising the nucleic acid sequence set forth in SEQ ID NO:1 and at Genbank Accession number U20532. In one embodiment, the present invention provides an Nrf2 gene comprising the nucleic acid sequence set forth by nucleotides 298 to 2043 in SEQ ID NO:1 and at Genbank Accession number U20532.

In one embodiment, the present invention provides an Nrf2 gene comprising the nucleic acid sequence set forth in SEQ ID NO:3 and at Genbank Accession number NM_{—}006164. In one embodiment, the present invention provides an Nrf2 gene comprising the nucleic acid sequence set forth by nucleotides 40 to 1809 in SEQ ID NO:3 and at Genbank Accession number NM_{—}006164.

In one embodiment, the present invention provides a Nrf2 gene comprising a nucleic acid sequence having at least about 90% identity to the nucleic acid sequence set forth in SEQ ID NO:1 and at Genbank Accession number U20532. In one embodiment, the present invention provides an Nrf2 gene comprising a nucleic acid sequence having at least about 90% identity to the nucleic acid sequence set forth by nucleotides 298 to 2043 in SEQ ID NO:1 and at Genbank Accession number U20532.

In one embodiment, the present invention provides a Nrf2 gene comprising a nucleic acid sequence having at least about 90% identity to the nucleic acid sequence set forth in SEQ ID NO:3 and at Genbank Accession number NM_{—}006164. In one embodiment, the present invention provides an Nrf2 gene comprising a nucleic acid sequence having at least about 90% identity to the nucleic acid sequence set forth by nucleotides 40 to 1809 in SEQ ID NO:3 and at Genbank Accession number NM_{—}006164.

In one embodiment, the present invention provides an Nrf2 gene comprising a nucleic acid that hybridizes under high stringency conditions to a nucleic acid comprising the nucleic acid sequence set forth in SEQ ID NO:1 and at Genbank Accession number U20532.

In one embodiment, the present invention provides an Nrf2 gene comprising a nucleic acid that hybridizes under high stringency conditions to a nucleic acid comprising the nucleic acid sequence set forth in SEQ ID NO:3 and at Genbank Accession number NM_{—}006164.

In one embodiment, the present invention provides an Nrf2 gene comprising the nucleic acid sequence set forth by nucleotides 1716 to 1850 in SEQ ID NO:1 and at Genbank Accession number U20532. In one embodiment, the present invention provides an Nrf2 gene comprising a nucleic acid which hybridizes under high stringency conditions to a nucleic acid comprising the nucleic acid sequence set forth by nucleotides 1716 to 1850 in SEQ ID NO:1 and at Genbank Accession number U20532. In one embodiment, the present invention provides an Nrf2 gene comprising a nucleic acid sequence having at least about 90% identity to the nucleic acid sequence set forth by nucleotides 1716 to 1850 in SEQ ID NO:1 and at Genbank Accession number U20532.

In one embodiment, the present invention provides an Nrf2 gene comprising the nucleic acid sequence set forth by nucleotides 1482 to 1616, more preferably 1482 to 1550, in SEQ ID NO:3 and at Genbank Accession number NM_{—}006164. In one embodiment, the present invention provides an Nrf2 gene comprising a nucleic acid which hybridizes under high stringency conditions to a nucleic acid comprising the nucleic acid sequence set forth by nucleotides 1482 to 1616, more preferably 1482 to 1550, in SEQ ID NO:3 and at Genbank Accession number NM_{—}006164. In one embodiment, the present invention provides an Nrf2 gene comprising a nucleic acid sequence having at least about 90% identity to the nucleic acid sequence set forth by nucleotides 1482 to 1616, more preferably 1482 to 1550, in SEQ ID NO:3 and at Genbank Accession number NM_{—}006164.

In one embodiment, the present invention provides an Nrf2 gene comprising a nucleic acid sequence that encodes an Nrf2 protein comprising the amino acid sequence set forth in SEQ ID NO:2 and at Genbank Accession Number AAA68291.

In one embodiment, the present invention provides an Nrf2 gene comprising a nucleic acid sequence that encodes an Nrf2 protein comprising the amino acid sequence set forth in SEQ ID NO:4 and at Genbank Accession Number NP_{—}006155.

In one embodiment, the present invention provides an Nrf2 gene comprising a nucleic acid sequence encoding an Nrf2 protein comprising the amino acid sequence set forth by amino acids 474 to 518 in SEQ ID NO:2 and at Genbank Accession Number AAA68291.

In one embodiment, the present invention provides an Nrf2 gene comprising a nucleic acid sequence encoding an Nrf2 protein comprising the amino acid sequence set forth by amino acids 482 to 526, more preferably 482 to 504, in SEQ ID NO:4 and at Genbank Accession Number NP_{—}006155.

In one embodiment, the present invention provides an Nrf2 gene comprising a nucleic acid sequence encoding an Nrf2 protein comprising the amino acid sequence set forth in SEQ ID NO:2 and at Genbank Accession Number AAA68291, except for lacking a fragment of the amino acid sequence set forth by amino acids 474 to 518 in SEQ ID NO:2 and at Genbank Accession Number AAA68291.

In one embodiment, the present invention provides an Nrf2 gene comprising a nucleic acid sequence encoding an Nrf2 protein comprising the amino acid sequence set forth in SEQ ID NO:4 and at Genbank Accession Number NP_{—}006155, except for lacking a fragment of the amino acid sequence set forth by amino acids 482 to 526, more preferably 482 to 504, in SEQ ID NO:4 and at Genbank Accession Number NP_{—}006155.

In one embodiment, the present invention provides Nrf2 proteins encoded by Nrf2 nucleic acids as described herein.

In its native form, Nrf2 protein is an intracellular protein. Intracellular proteins may be found in the cytoplasm and/or in the nucleus. Intracellular proteins are involved in all aspects of cellular function and replication (including, for example, signaling pathways); aberrant expression of such proteins results in unregulated or disregulated cellular processes. For example, many intracellular proteins have enzymatic activity such as protein kinase activity, phosphatidyl inositol-conjugated lipid kinase activity, protein phosphatase activity, phosphatidyl inositol-conjugated lipid phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

Intracellular proteins found in the nucleus include DNA-binding transcription regulatory factors, or transcription factors. These proteins typically bind to specific nucleic acid sequences located in the regulatory regions of target genes and modulate the transcription of these target genes. Without being bound by theory, DNA-binding transcription factors can act, directly or indirectly, on a number of factors associated with the transcriptional apparatus including RNA polymerases and basal transcription factors. DNA binding transcription factors can also act at a number of stages during assembly of the transcriptional apparatus, initiation of transcription, and transcript elongation.

An increasingly appreciated concept in characterizing intracellular proteins is the presence in the proteins of one or more motifs for which defined functions have been attributed. In addition to the highly conserved sequences found in the enzymatic domain of proteins, highly conserved sequences have been identified that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, PDZ domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate.

Common protein motifs have also been identified among transcription factors including the basic helix-loop-helix, basic leucine zipper, zinc finger and homeodomain motifs.

It is recognized that through the use of recombinant DNA techniques, Nrf2 sequences can be made to be transmembrane proteins. Transmembrane proteins are molecules that span the phospholipid bilayer of a cell. They generally include approximately 20 consecutive hydrophobic amino acids that may be followed by charged amino acids. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

It will also be appreciated by those in the art that a transmembrane protein can be made soluble by removing transmembrane sequences, for example through recombinant methods. Furthermore, transmembrane proteins that have been made soluble can be made to be secreted by adding an appropriate signal sequence through recombinant means.

It is further recognized that Nrf2 proteins can be made to be secreted proteins though recombinant methods. Secretion can be either constitutive or regulated. Secreted proteins have a signal peptide or signal sequence that targets the molecule to the secretory pathway.

In another preferred embodiment, the Nrf2 proteins are nuclear proteins, preferably transcription factors. Transcription factors are involved in numerous physiological events and act by regulating gene expression at the transcriptional level. Transcription factors often serve as nodal points of regulation controlling multiple genes. They are capable of effecting a multifarious change in gene expression and can integrate many convergent signals to effect such a change. Transcription factors are often regarded as “master regulators” of a particular cellular state or event. Accordingly, transcription factors have often been found to faithfully mark a particular cell state, a quality which makes them attractive for use as diagnostic markers. In addition, because of their important role as coordinators of patterns of gene expression associated with particular cell states, transcription factors are attractive therapeutic targets. Intervention at the level of transcriptional regulation allows one to effectively target multiple genes associated with a dysfunction which fall under the regulation of a “master regulator” or transcription factor.

In one embodiment, an Nrf2 sequence can be identified by substantial nucleic acid sequence identity or homology to the Nrf2 nucleic acid sequence set forth in SEQ ID NO:1 and at Genbank Accession number U20532.

In another embodiment, an Nrf2 sequence can be identified by substantial nucleic acid sequence identity or homolgy to the Nrf2 nucleic acid sequence set forth in SEQ ID NO:3 and at Genbank Accession number NM_{—}006164.

In one embodiment, an Nrf2 sequence can be identified by substantial amino acid sequence identity or homology to the Nrf2 amino acid sequence set forth in SEQ ID NO:2 and at Genbank Accession number AAA68291.

In another embodiment, an Nrf2 sequence can be identified by substantial amino acid sequence identity or homology to the Nrf2 amino acid sequence set forth in SEQ ID NO:4 and at Genbank Accession number NP_{—}006155.

Such homology can be based upon the overall nucleic acid or amino acid sequence, and is generally determined as outlined below, using either homology and identity programs or hybridization conditions.

As used herein, a nucleic acid is an “Nrf2 nucleic acid” if the overall homology of the nucleic acid sequence to SEQ ID NO:1 or SEQ ID NO:3 is preferably greater than about 75%, more preferably greater than about 80%, even more preferably greater than about 85% and most preferably greater than 90%. In some embodiments the homology will be as high as about 93 to 95 or 98%. In one embodiment, the sequences are naturally occurring allelic variants of the nucleic acid sequence set forth in SEQ ID NO:1 or SEQ ID NO:3. In another embodiment, the sequences are variants of the nucleic acid sequence set forth in SEQ ID NO:1 or SEQ ID NO:3 as further described herein.

Homology in this context means sequence similarity or identity, with identity being preferred. A preferred comparison for homology purposes is to compare the sequence containing sequencing errors to the correct sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996); http://blast.wustl]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Thus, “percent (%) nucleic acid sequence identity” is defined as the percentage of nucleotide residues in a candidate sequence that are identical with the nucleotide residues of the nucleic acid set forth in SEQ ID NO:1 or SEQ ID NO:3. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer nucleotides than those of the nucleic acid sequence set forth in SEQ ID NO:1 or SEQ ID NO:3, it is understood that the percentage of homology will be determined based on the number of homologous nucleosides in relation to the total number of nucleosides. Thus, for example, homology of sequences shorter than those of the sequences identified herein and as discussed below, will be determined using the number of nucleosides in the shorter sequence.

In one embodiment, the nucleic acid homology is determined through hybridization studies. Thus, for example, nucleic acids which hybridize under high stringency to the nucleic acids identified in the tables, or their complements, are considered Nrf2 sequences. High stringency conditions are known in the art; see for example Maniatis et al., Molecular Cloning: A Laboratory Manual, 2d Edition, 1989, and Current Protocols in Molecular Biology, Eds. F. M. Ausubel et al., NY, Greene Publ. Associates and Wiley Interscience, J. Wiley, 1988) both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

In another embodiment, less stringent hybridization conditions are used; for example, moderate or low stringency conditions may be used, as are known in the art; see Maniatis and Ausubel, supra, and Tijssen, supra.

In addition, in one embodiment, the Nrf2 nucleic acid sequences of the invention are fragments of larger genes, i.e. they are nucleic acid segments. Alternatively, the Nrf2 nucleic acid sequences can serve as indicators of oncogene position, for example, the Nrf2 sequence may be an enhancer that activates a protooncogene. “Genes” in this context includes coding regions, non-coding regions, and mixtures of coding and non-coding regions. Accordingly, as will be appreciated by those in the art, using the sequences provided herein, additional sequences of the Nrf2 genes can be obtained, using techniques well known in the art for cloning either longer sequences or the full length sequences; see Maniatis et al., and Ausubel, et al., supra, hereby expressly incorporated by reference. In general, this is done using PCR, for example, kinetic PCR.

Once the Nrf2 nucleic acid is identified, it can be cloned and, if necessary, its constituent parts recombined to form the entire Nrf2 nucleic acid. Once isolated from its natural source, e.g., contained within a plasmid or other vector or excised therefrom as a linear nucleic acid segment, the recombinant Nrf2 nucleic acid can be further used as a probe to identify and isolate other Nrf2 nucleic acids, for example additional coding regions. It can also be used as a “precursor” nucleic acid to make modified or variant Nrf2 nucleic acids and proteins.

The Nrf2 nucleic acids of the present invention are used in several ways. In a first embodiment, nucleic acid probes to the Nrf2 nucleic acids are made and attached to biochips to be used in screening and diagnostic methods, as outlined below, or for administration, for example for gene therapy and/or antisense applications. Alternatively, the Nrf2 nucleic acids that include coding regions of Nrf2 proteins can be put into expression vectors for the expression of Nrf2 proteins, again either for screening purposes or for administration to a patient.

In a preferred embodiment, nucleic acid probes, to Nrf2 nucleic acids comprising the nucleic acid sequence set forth in SEQ ID NO:1 or SEQ ID NO:3, or complements thereof, or fragments thereof or complements of fragments thereof, are made. In another preferred embodiment, nucleic acid probes, to Nrf2 nucleic acid variants having nucleic acid sequence identity of at least about 90% to SEQ ID NO:1 or SEQ ID NO:3, or complements thereof, or fragments thereof or complements of fragments thereof, are made. In another preferred embodiment, nucleic acid probes, to Nrf2 nucleic acid variants capable of hybridizing to nucleic acids comprising the nucleic acid sequence set forth in SEQ ID NO:1 or SEQ ID NO:3, or complements thereof, or fragments thereof or complements of fragments thereof, are made. The nucleic acid probes attached to the biochip are designed to be substantially complementary to the Nrf2 nucleic acid or Nrf2 nucleic acid variant, i.e. the target sequence (either the target sequence of the sample or to other probe sequences, for example in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e. have some sequence in common), or separate.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant any material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, etc. In general, the substrates allow optical detection and do not appreciably fluoresce.

In a preferred embodiment, the surface of the biochip and the probe may be derivatized with chemical functional groups for subsequent attachment of the two. Thus, for example, the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, for example using linkers as are known in the art; for example, homo-or hetero-bifunctional linkers as are well known (see 1994 Pierce Chemical Company catalog, technical section on cross-linkers, pages 155-200, incorporated herein by reference). In addition, in some cases, additional linkers, such as alkyl groups (including substituted and heteroalkyl groups) may be used.

In this embodiment, the oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In an additional embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affimetrix GeneChip™ technology.

In addition to the solid-phase technology represented by biochip arrays, gene expression can also be quantified using liquid-phase arrays. One such system is kinetic polymerase chain reaction (PCR). Kinetic PCR allows for the simultaneous amplification and quantification of specific nucleic acid sequences. The specificity is derived from synthetic oligonucleotide primers designed to preferentially adhere to single-stranded nucleic acid sequences bracketing the target site. This pair of oligonucleotide primers form specific, non-covalently bound complexes on each strand of the target sequence. These complexes facilitate in vitro transcription of double-stranded DNA in opposite orientations. Temperature cycling of the reaction mixture creates a continuous cycle of primer binding, transcription, and re-melting of the nucleic acid to individual strands. The result is an exponential increase of the target dsDNA product. This product can be quantified in real time either through the use of an intercalating dye or a sequence specific probe. SYBR® Greene I, is an example of an intercalating dye, that preferentially binds to dsDNA resulting in a concomitant increase in the fluorescent signal. Sequence specific probes, such as used with TaqMan® technology, consist of a fluorochrome and a quenching molecule covalently bound to opposite ends of an oligonucleotide. The probe is designed to selectively bind the target DNA sequence between the two primers. When the DNA strands are synthesized during the PCR reaction, the fluorochrome is cleaved from the probe by the exonuclease activity of the polymerase resulting in signal dequenching. The probe signaling method can be more specific than the intercalating dye method, but in each case, signal strength is proportional to the dsDNA product produced. Each type of quantification method can be used in multi-well liquid phase arrays with each well representing primers and/or probes specific to nucleic acid sequences of interest. When used with messenger RNA preparations of tissues or cell lines, and an array of probe/primer reactions can simultaneously quantify the expression of multiple gene products of interest. See Germer, S., et al., Genome Res. 10:258-266 (2000); Heid, C. A., et al., Genome Res. 6, 986-994 (1996).

In a preferred embodiment, Nrf2 nucleic acids encoding Nrf2 proteins are used to make a variety of expression vectors to express Nrf2 proteins which can then be used in screening assays, as described below. The expression vectors may be either self-replicating extrachromosomal vectors or vectors which integrate into a host genome. Generally, these expression vectors include transcriptional and translational regulatory nucleic acid operably linked to the nucleic acid encoding the Nrf2 protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice. The transcriptional and translational regulatory nucleic acid will generally be appropriate to the host cell used to express the Nrf2 protein; for example, transcriptional and translational regulatory nucleic acid sequences from Bacillus are preferably used to express the Nrf2 protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The Nrf2 proteins of the present invention are produced by culturing a host cell transformed with an expression vector containing nucleic acid encoding an Nrf2 protein, under the appropriate conditions to induce or cause expression of the Nrf2 protein. The conditions appropriate for Nrf2 protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect, plant and animal cells, including mammalian cells. Of particular interest are Drosophila melanogaster cells, Saccharomyces cerevisiae and other yeasts, E. coli, Bacillus subtilis, Sf9 cells, C129 cells, 293 cells, Neurospora, BHK, CHO, COS, HeLa cells, THP1 cell line (a macrophage cell line) and human cells and cell lines.

In a preferred embodiment, the Nrf2 proteins are expressed in mammalian cells. Mammalian expression systems are also known in the art, and include retroviral systems. A preferred expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenylation signals include those derived form SV40.

Methods for introducing exogenous nucleic acid into mammalian hosts, as well as other hosts, are well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, Nrf2 protein is expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the Nrf2 protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, Nrf2 proteins are produced in insect cells. Expression vectors for the transformation of insect cells, and in particular, baculovirus-based expression vectors, are well known in the art.

In a preferred embodiment, Nrf2 protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The Nrf2 protein may also be made as a fusion protein, using techniques well known in the art. Such fusion proteins may be used for example, for the creation of monoclonal antibodies. If the desired epitope is small, the Nrf2 protein may be fused to a carrier protein to form an immunogen. Alternatively, the Nrf2 protein may be made as a fusion protein to increase expression, or for other reasons. For example, when the Nrf2 protein is an Nrf2 peptide, the nucleic acid encoding the peptide may be linked to other nucleic acid for expression purposes.

In one embodiment, the Nrf2 nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the Nrf2 nucleic acids, proteins and antibodies at any position. For example, the label should be capable of producing, either directly or indirectly, a detectable signal. The detectable moiety may be a radioisotope, such as ³H, ¹⁴C, ³²p, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Accordingly, the present invention also provides Nrf2 protein sequences. An Nrf2 protein of the present invention may be identified in several ways. “Protein” in this sense includes proteins, polypeptides, and peptides. As will be appreciated by those in the art, the nucleic acid sequences of the invention can be used to generate protein sequences. There are a variety of ways to do this, including cloning the entire gene and verifying its frame and amino acid sequence, or by comparing it to known sequences to search for homology to provide a frame, assuming the Nrf2 protein has homology to some protein in the database being used. Generally, the nucleic acid sequences are input into a program that will search all three frames for homology. This is done in a preferred embodiment using the following NCBI Advanced BLAST parameters. The program is blastx or blastn. The database is nr. The input data is as “Sequence in FASTA format”. The organism list is “none”. The “expect” is 10; the filter is default. The “descriptions” is 500, the “alignments” is 500, and the “alignment view” is pairwise. The “Query Genetic Codes” is standard (1). The matrix is BLOSUM62; gap existence cost is 11, per residue gap cost is 1; and the lambda ratio is 0.85 default. This results in the generation of a putative protein sequence.

Also included within one embodiment of Nrf2 proteins are amino acid variants of the naturally occurring sequences, as determined herein. Preferably, the variants are preferably greater than about 75% homologous to the wild-type sequence, more preferably greater than about 80%, even more preferably greater than about 85% and most preferably greater than 90%. In some embodiments the homology will be as high as about 93 to 95 or 98%. As for nucleic acids, homology in this context means sequence similarity or identity, with identity being preferred. This homology will be determined using standard techniques known in the art as are outlined above for the nucleic acid homologies.

Nrf2 proteins of the present invention may be shorter or longer than the wild type amino acid sequences. Thus, in a preferred embodiment, included within the definition of Nrf2 proteins are portions or fragments of the wild type sequences herein. In addition, as outlined above, the Nrf2 nucleic acids of the invention may be used to obtain additional coding regions, and thus additional protein sequence, using techniques known in the art.

In a preferred embodiment, the Nrf2 proteins are derivative or variant Nrf2 proteins as compared to the wild-type sequence. That is, as outlined more fully below, the derivative Nrf2 peptide will contain at least one amino acid substitution, deletion or insertion, with amino acid substitutions being particularly preferred. The amino acid substitution, insertion or deletion may occur at any residue within the Nrf2 peptide.

Also included in an embodiment of Nrf2 proteins of the present invention are amino acid sequence variants. These variants fall into one or more of three classes: substitutional, insertional or deletional variants. These variants ordinarily are prepared by site specific mutagenesis of nucleotides in the DNA encoding the Nrf2 protein, using cassette or PCR mutagenesis or other techniques well known in the art, to produce DNA encoding the variant, and thereafter expressing the DNA in recombinant cell culture as outlined above. However, variant Nrf2 protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the Nrf2 protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed Nrf2 variants screened for the optimal combination of desired activity. Techniques for making substitution mutations at predetermined sites in DNA having a known sequence are well known, for example, M13 primer mutagenesis and LAR mutagenesis. Screening of the mutants is done using assays of Nrf2 protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the Nrf2 protein are desired, substitutions are generally made in accordance with the following chart:

Chart I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the Nrf2 proteins as needed. Alternatively, the variant may be designed such that the biological activity of the Nrf2 protein is altered. For example, glycosylation sites may be altered or removed, dominant negative mutations created, etc.

Covalent modifications of Nrf2 polypeptides are included within the scope of this invention, for example for use in screening. One type of covalent modification includes reacting targeted amino acid residues of an Nrf2 polypeptide with an organic derivatizing agent that is capable of reacting with selected side chains or the N-or C-terminal residues of an Nrf2 polypeptide. Derivatization with bifunctional agents is useful, for instance, for crosslinking Nrf2 to a water-insoluble support matrix or surface for use in the method for purifying anti-LA antibodies or screening assays, as is more fully described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the α-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group, and sulfonation of residues on secreted proteins.

Another type of covalent modification of the Nrf2 polypeptide included within the scope of this invention comprises altering the native glycosylation pattern of the polypeptide. “Altering the native glycosylation pattern” is intended for purposes herein to mean deleting one or more carbohydrate moieties found in native sequence Nrf2 polypeptide, and/or adding one or more glycosylation sites that are not present in the native sequence Nrf2 polypeptide.

Addition of glycosylation sites to Nrf2 polypeptides may be accomplished by altering the amino acid sequence thereof. The alteration may be made, for example, by the addition of, or substitution by, one or more serine or threonine residues to the native sequence Nrf2 polypeptide (for O-linked glycosylation sites). The Nrf2 amino acid sequence may optionally be altered through changes at the DNA level, particularly by mutating the DNA encoding the Nrf2 polypeptide at preselected bases such that codons are generated that will translate into the desired amino acids.

Another means of increasing the number of carbohydrate moieties on the Nrf2 polypeptide is by chemical or enzymatic coupling of glycosides to the polypeptide. Such methods are described in the art, e.g., in WO 87/05330 published Sep. 11, 1987, and in Aplin and Wriston, LA Crit. Rev. Biochem., pp. 259-306 (1981).

Removal of carbohydrate moieties present on the Nrf2 polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of Nrf2 comprises linking the Nrf2 polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Nrf2 polypeptides of the present invention may also be modified in a way to form chimeric molecules comprising an Nrf2 polypeptide fused to another, heterologous polypeptide or amino acid sequence. In one embodiment, such a chimeric molecule comprises a fusion of an Nrf2 polypeptide with a tag polypeptide which provides an epitope to which an anti-tag antibody can selectively bind. The epitope tag is generally placed at the amino-or carboxyl-terminus of the Nrf2 polypeptide, although internal fusions may also be tolerated in some instances. The presence of such epitope-tagged forms of an Nrf2 polypeptide can be detected using an antibody against the tag polypeptide. Also, provision of the epitope tag enables the Nrf2 polypeptide to be readily purified by affinity purification using an anti-tag antibody or another type of affinity matrix that binds to the epitope tag. In an alternative embodiment, the chimeric molecule may comprise a fusion of an Nrf2 polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Also included with the definition of Nrf2 protein in one embodiment are other Nrf2 proteins of the Nrf2 family, and Nrf2 proteins from other organisms, which are cloned and expressed as outlined below. Thus, probe or degenerate polymerase chain reaction (PCR) primer sequences may be used to find other related Nrf2 proteins from humans or other organisms. As will be appreciated by those in the art, particularly useful probe and/or PCR primer sequences include the unique areas of the Nrf2 nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art.

In addition, as is outlined herein, Nrf2 proteins can be made that are longer than those encoded by the nucleic acids of the tables, for example, by the elucidation of additional sequences, the addition of epitope or purification tags, the addition of other fusion sequences, etc.

Nrf2 proteins may also be identified as being encoded by Nrf2 nucleic acids. Thus, Nrf2 proteins are encoded by nucleic acids that will hybridize to the sequences of the sequence listings, or their complements, as outlined herein.

In one embodiment, the present invention provides an LA protein referred to herein as Nrf2 which comprises the amino acid sequence set forth in SEQ ID NO:2 and at Genbank accession number AAA68291, and which is encoded by the nucleic acid sequence set forth by nucleotides 298 to 2043 in SEQ ID NO:1 and at Genbank accession number U20532.

In one embodiment, the present invention provides an LA protein referred to herein as Nrf2 which comprises the amino acid sequence set forth in SEQ ID NO:4 and at Genbank accession number NP_{—}006155, and which is encoded by the nucleic acid sequence set forth by nucleotides 40 to 1809 in SEQ ID NO:3 and at Genbank accession number NM_{—}006164.

In one embodiment, the present invention provides an Nrf2 protein encoded by a nucleic acid which hybridizes under high stringency conditions to a nucleic acid comprising the nucleic acid sequence set forth in SEQ ID NO:1 and at Genbank accession number U20532.

In one embodiment, the present invention provides an Nrf2 protein encoded by a nucleic acid which hybridizes under high stringency conditions to a nucleic acid comprising the nucleic acid sequence set forth in SEQ ID NO:3 and at Genbank accession number NM_{—}006164.

In one embodiment, the present invention provides an Nrf2 protein encoded by a nucleic acid which comprises a nucleic acid sequence having at least about 90% identity to the nucleic acid sequence set forth in SEQ ID NO:1 and at Genbank accession number U20532.

In one embodiment, the present invention provides an Nrf2 protein encoded by a nucleic acid which comprises a nucleic acid sequence having at least about 90% identity to the nucleic acid sequence set forth in SEQ ID NO:3 and at Genbank accession number NM_{—}006164.

In one embodiment, the present invention provides an Nrf2 protein encoded by a nucleic acid which comprises a nucleic acid sequence having at least about 90% identity to the nucleic acid sequence set forth by nucleotides 298-2043 in SEQ ID NO:1 and at Genbank accession number U20532.

In one embodiment, the present invention provides an Nrf2 protein encoded by a nucleic acid which comprises a nucleic acid sequence having at least about 90% identity to the nucleic acid sequence set forth by nucleotides 40-1809 in SEQ ID NO:3 and at Genbank accession number NM_{—}006164.

In one embodiment, the present invention provides an Nrf2 protein comprising a basic leucine zipper domain encoded by the nucleic acid sequence set forth by nucleotides 1716 to 1850 in SEQ ID NO:1 and at Genbank Accession Number U20532.

In one embodiment, the present invention provides an Nrf2 protein comprising a basic leucine zipper domain encoded by the nucleic acid sequence set forth by nucleotides 1482 to 1616, more preferably 1482 to 1550, in SEQ ID NO:3 and at Genbank Accession Number NM_{—}006164.

In one embodiment, the present invention provides an Nrf2 protein comprising a basic leucine zipper domain encoded by a nucleic acid that hybridizes under high stringency conditions to a nucleic acid comprising the nucleic acid sequence set forth by nucleotides 1716 to 1850 in SEQ ID NO:1 and at Genbank Accession Number U20532.

In one embodiment, the present invention provides an Nrf2 protein comprising a basic leucine zipper domain encoded by a nucleic acid that hybridizes under high stringency conditions to a nucleic acid comprising the nucleic acid sequence set forth by nucleotides 1482 to 1616, more preferably 1482 to 1550, in SEQ ID NO:3 and at Genbank Accession Number NM_{—}006164.

In one embodiment, the present invention provides an Nrf2 protein comprising a basic leucine zipper domain encoded by a nucleic acid comprising a nucleic acid sequence having at least about 90% identity to the nucleic acid sequence set forth by nucleotides 1716 to 1850 in SEQ ID NO:1 and at Genbank Accession Number U20532.

In one embodiment, the present invention provides an Nrf2 protein comprising a basic leucine zipper domain encoded by a nucleic acid comprising a nucleic acid sequence having at least about 90% identity to the nucleic acid sequence set forth by nucleotides 1482 to 1616, more preferably 1482 to 1550, in SEQ ID NO:3 and at Genbank Accession Number NM_{—}006164.

In one embodiment, the present invention provides an Nrf2 protein comprising the amino acid sequence set forth in SEQ ID NO:2 and at Genbank Accession number AAA68291.

In one embodiment, the present invention provides an Nrf2 protein comprising the amino acid sequence set forth in SEQ ID NO:4 and at Genbank Accession number NP_{—}006155.

In one embodiment, the present invention provides an Nrf2 protein comprising an amino acid sequence having at least about 90% identity to the amino acid sequence set forth in SEQ ID NO:2 and at Genbank Accession Number AAA68291.

In one embodiment, the present invention provides an Nrf2 protein comprising an amino acid sequence having at least about 90% identity to the amino acid sequence set forth in SEQ ID NO:4 and at Genbank Accession Number NP_{—}006155.

In one embodiment, the present invention provides an Nrf2 protein comprising the amino acid sequence set forth by amino acids 474 to 518 in SEQ ID NO:2 and at Genbank Accession Number AAA68291.

In one embodiment, the present invention provides an Nrf2 protein comprising the amino acid sequence set forth by amino acids 482 to 526, more preferably 482 to 504, in SEQ ID NO:4 and at Genbank Accession Number NP_{—}006155.

In one embodiment, the present invention provides an Nrf2 protein comprising an amino acid sequence having at least 90% identity to the amino acid sequence set forth by amino acids 474 to 518 in SEQ ID NO:2 and at Genbank Accession Number AAA68291.

In one embodiment, the present invention provides an Nrf2 protein comprising an amino acid sequence having at least 90% identity to the amino acid sequence set forth by amino acids 482 to 526, more preferably 482 to 504, in SEQ ID NO:4 and at Genbank Accession Number NP_{—}006155.

In one embodiment, the present invention provides an Nrf2 protein comprising the amino acid sequence set forth in SEQ ID NO:2 and at Genbank accession number AAA68291, except for lacking a fragment of the amino acid sequence set forth by amino acids 474 to 518 in SEQ ID NO:2 and at Genbank accession number AAA68291.

In one embodiment, the present invention provides an Nrf2 protein comprising the amino acid sequence set forth in SEQ ID NO:4 and at Genbank accession number NP_{—}006155, except for lacking a fragment of the amino acid sequence set forth by amino acids 482 to 526, more preferably 482 to 504, in SEQ ID NO:4 and at Genbank Accession Number NP_{—}006155.

In one embodiment, the present invention provides an Nrf2 protein variant which lacks a basic amino acid sequence normally involved in DNA binding in a protein comprising the amino acid sequence set forth in SEQ ID NO:2 and at Genbank accession number AAA68291.

In another embodiment, the present invention provides an Nrf2 protein variant which lacks a basic amino acid sequence normally involved in DNA binding in a protein comprising the amino acid sequence set forth in SEQ ID NO:4 and at Genbank accession number NP_{—}006155.

In one embodiment, the present invention provides an Nrf2 protein variant which lacks an amino acid sequence normally involved in DNA binding and protein-protein interaction in a protein comprising the amino acid sequence set forth in SEQ ID NO:2 and at Genbank accession number AAA68291.

In one embodiment, the present invention provides an Nrf2 protein variant which lacks an amino acid sequence normally involved in DNA binding and protein-protein interaction in a protein comprising the amino acid sequence set forth in SEQ ID NO:4 and at Genbank accession number NP_{—}006155.

In one embodiment, the present invention provides an Nrf2 protein variant which lacks an amino acid sequence, preferably an acidic sequence, which is normally involved in transcriptional regulation in a protein comprising the amino acid sequence set forth in SEQ ID NO:2 and at Genbank accession number AAA68291.

In one embodiment, the present invention provides an Nrf2 protein variant which lacks an amino acid sequence, preferably an acidic sequence, which is normally involved in transcriptional regulation in a protein comprising the amino acid sequence set forth in SEQ ID NO:4 and accession number NP_{—}006155.

In one embodiment, the present invention provides an Nrf2 protein variant which lacks an amino acid sequence normally involved in transcriptional regulation and protein-protein interaction in a protein comprising the amino acid sequence set forth in SEQ ID NO:2 and at AAA68291.

In one embodiment, the present invention provides an Nrf2 protein variant which lacks an amino acid sequence normally involved in transcriptional regulation and protein-protein interaction in a protein comprising the amino acid sequence set forth in SEQ ID NO:4 and at Genbank accession number NP_{—}006155.

It is recognized that such variant Nrf2 proteins may serve as dominant negative effectors of Nrf2 protein function.

In a preferred embodiment, the invention provides Nrf2 antibodies. In a preferred embodiment, when the Nrf2 protein is to be used to generate antibodies, for example for immunotherapy, the Nrf2 protein should share at least one epitope or determinant with the full length protein. By “epitope” or “determinant” herein is meant a portion of a protein which will generate and/or bind an antibody or T-cell receptor in the context of MHC. Thus, in most instances, antibodies made to a smaller Nrf2 protein will be able to bind to the full length protein. In a preferred embodiment, the epitope is unique; that is, antibodies generated to a unique epitope show little or no cross-reactivity.

In one embodiment, the term “antibody” includes antibody fragments, as are known in the art, including Fab, Fab₂, single chain antibodies (Fv for example), chimeric antibodies, etc., either produced by the modification of whole antibodies or those synthesized de novo using recombinant DNA technologies.

Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the tables or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by the nucleic acid sequence set forth in SEQ ID NO:1 or fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens. In the present case, one of the binding specificities is for a protein encoded by the nucleic acid sequence set forth in SEQ ID NO:1 or SEQ ID NO:3, or a fragment thereof, the other one is for any other antigen, and preferably for a cell-surface protein or receptor or receptor subunit, preferably one that is tumor specific.

In a preferred embodiment, the antibodies to Nrf2 are capable of reducing or eliminating the biological function of Nrf2, as is described below. That is, the addition of anti-Nrf2 antibodies (either polyclonal or preferably monoclonal) to Nrf2 (or cells containing Nrf2) may reduce or eliminate the Nrf2 activity. Generally, at least a 25% decrease in activity is preferred, with at least about 50% being particularly preferred and about a 95-100% decrease being especially preferred.

In a preferred embodiment the antibodies to the Nrf2 proteins are humanized antibodies. Humanized forms of non-human (e.g., murine) antibodies are chimeric molecules of immunoglobulins, immunoglobulin chains or fragments thereof (such as Fv, Fab, Fab′, F(ab′)₂ or other antigen binding sub-sequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework residues (FR) regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies [Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

By immunotherapy is meant treatment of lymphoma with an antibody raised against an Nrf2 protein. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen.

In another preferred embodiment, Nrf2 transcription factors which bind specific nucleic acid sequences, preferably the sequence GCTGAGTCATGATGAGTCA, or variants thereof having at least about 90% sequence identity, more preferably about 95%, more preferably 98%, more preferably 99%, more preferably 100%, are inhibited by antibodies raised against Nrf2 proteins. Without being bound by theory, the antibody binds to the Nrf2 transcription factor and affects its ability to bind DNA and/or regulate transcription.

In a preferred embodiment, the Nrf2 transcription factor comprises the amino acid sequence set forth in SEQ ID NO:2 and at Genbank Accession number AAA68291, or a portion thereof.

In another preferred embodiment, the Nrf2 transcription factor comprises the amino acid sequence set forth in SEQ ID NO:4 and at Genbank Accession number NP_{—}006155, or a portion thereof.

In another preferred embodiment, the Nrf2 transcription factor comprises a basic leucine zipper motif comprising the amino acid sequence set forth by amino acids 474 to 518 in SEQ ID NO:2 and at Genbank accession number AAA68291.

In another preferred embodiment, the Nrf2 transcription factor comprises a basic leucine zipper motif comprising the amino acid sequence set forth by amino acids 482 to 526, more preferably 482 to 504, in SEQ ID NO:4 and at Genbank accession number NP_{—}006155.

In another preferred embodiment, the Nrf2 transcription factor comprises a basic leucine zipper motif comprising an amino acid sequence having at least about 90% identity to the amino acid sequence set forth by amino acids 474 to 518 in SEQ ID NO:2 and at Genbank accession number AAA68291.

In another preferred embodiment, the Nrf2 transcription factor comprises a basic leucine zipper motif comprising an amino acid sequence having at least about 90% identity to the amino acid sequence set forth by amino acids 482 to 526, more preferably 482 to 504, in SEQ ID NO:4 and at Genbank accession number NP_{—}006155.

In another preferred embodiment, the Nrf2 transcription factor comprises a basic leucine zipper motif comprising an amino acid sequence encoded by the nucleic acid sequence set forth by nucleotides 1716 to 1850 in SEQ ID NO:1 and at Genbank Accession No. U20532.

In another preferred embodiment, the Nrf2 transcription factor comprises a basic leucine zipper motif comprising an amino acid sequence encoded by the nucleic acid sequence set forth by nucleotides 1482 to 1616, more preferably 1482 to 1550, in SEQ ID NO:3 and at Genbank Accession No. NM_{—}006164.

In another preferred embodiment, the Nrf2 transcription factor comprises a basic leucine zipper motif comprising an amino acid sequence encoded by a nucleic acid which will hybridize under high stringency conditions to a nucleic acid comprising the nucleic acid sequence set forth by nucleotides 1716 to 1850 in SEQ ID NO:1 and at Genbank Accession No. U20532.

In another preferred embodiment, the Nrf2 transcription factor comprises a basic leucine zipper motif comprising an amino acid sequence encoded by a nucleic acid which will hybridize under high stringency conditions to a nucleic acid comprising the nucleic acid sequence set forth by nucleotides 25 1482 to 1616, more preferably 1482 to 1550, in SEQ ID NO:3 and at Genbank Accession No. NM_{—}006164.

In another preferred embodiment, the Nrf2 transcription factor comprises a basic leucine zipper motif comprising an amino acid sequence encoded by a nucleic acid which comprises a nucleic acid sequence having at least about 90% identity to the nucleic acid sequence set forth by nucleotides 1716 to 1850 in SEQ ID NO:1 and at Genbank Accession No. U20532.

In another preferred embodiment, the Nrf2 transcription factor comprises a basic leucine zipper motif comprising an amino acid sequence encoded by a nucleic acid which comprises a nucleic acid sequence having at least about 90% identity to the nucleic acid sequence set forth by nucleotides 1482 to 1616, more preferably 1482 to 1550, in SEQ ID NO:3 and at Genbank Accession No. NM_{—}006164.

In a preferred embodiment, an antibody raised against an Nrf2 protein binds an amino acid sequence involved in DNA binding, for example, an amino acid sequence comprising the basic region of a basic leucine zipper domain-containing protein.

In a preferred embodiment, an antibody raised against an Nrf2 protein binds an amino acid sequence involved in transcriptional regulation, for example, an amino acid sequence comprising an acidic domain in a basic leucine zipper domain-containing protein.

In a preferred embodiment, an antibody raised against an Nrf2 protein binds an amino acid sequence involved in protein-protein interaction. For example, an amino acid sequence comprising the leucine zipper domain or an acidic domain in a basic leucine zipper domain-containing protein. In a preferred embodiment, such protein-protein interaction involves a component of the transcriptional apparatus. In a preferred embodiment, such protein-protein interaction involves a transcriptional cofactor. In a preferred embodiment, such protein-protein interaction involves a protein having a leucine zipper or a zinc finger.

By determining Nrf2 is important in mechanisms of cellular dysregulation underlying lymphoma, it will be recognized that the present application implicates genes having an Nrf2 binding sequence, and/or genes that are modulated by Nrf2 activity, in these cellular mechanisms.

In another preferred embodiment, the antibody is conjugated to a therapeutic moiety. In one aspect the therapeutic moiety is a small molecule that modulates the activity of the Nrf2 protein. In another aspect the therapeutic moiety modulates the activity of molecules associated with or in close proximity to the Nrf2 protein. The therapeutic moiety may inhibit enzymatic activity such as protease or protein kinase activity associated with lymphoma.

In a preferred embodiment, the therapeutic moiety may also be a cytotoxic agent. In this method, targeting the cytotoxic agent to tumor tissue or cells, results in a reduction in the number of afflicted cells, thereby reducing symptoms associated with lymphoma. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against Nrf2 proteins, or binding of a radionuclide to a chelating agent that has been covalently attached to the antibody.

Targeting the therapeutic moiety to transmembrane Nrf2 proteins not only serves to increase the local concentration of therapeutic moiety in the lymphoma, but also serves to reduce deleterious side effects that may be associated with the therapeutic moiety.

In another preferred embodiment, the Nrf2 protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the Nrf2 protein can be targeted within a cell, i.e., the nucleus, an antibody thereto contains a signal for that target localization, i.e., a nuclear localization signal.

The Nrf2 antibodies of the invention specifically bind to Nrf2 proteins. By “specifically bind” herein is meant that the antibodies bind to the protein with a binding constant in the range of at least 10⁻⁴-10⁻⁶ M⁻¹, with a preferred range being 10⁻⁷-10⁻⁹ M⁻¹.

In a preferred embodiment, the Nrf2 protein is purified or isolated after expression. Nrf2 proteins may be isolated or purified in a variety of ways known to those skilled in the art depending on what other components are present in the sample. Standard purification methods include electrophoretic including 2D electrophoresis, molecular, immunological and chromatographic techniques, including ion exchange, hydrophobic, affinity, and reverse-phase HPLC chromatography, and chromatofocusing. For example, the Nrf2 protein may be purified using a standard anti-Nrf2 antibody column. Ultrafiltration and diafiltration techniques, in conjunction with protein concentration, are also useful. For general guidance in suitable purification techniques, see Scopes, R., Protein Purification, Springer-Verlag, N.Y. (1982). The degree of purification necessary will vary depending on the use of the Nrf2 protein. In some instances no purification will be necessary.

Once expressed and purified if necessary, the Nrf2 proteins and nucleic acids are useful in a number of applications.

In one aspect, the level of expression of multiple genes is determined in different cell samples corresponding to different cellular states to produce a gene expression profile for an array of cellular states. A gene expression profile derived for a particular cell state is essentially a “finger print” of that cellular state. Cellular states characteristic of normal lymphoid tissue and characteristic of lymphoid tissue found in lymphomas are included. Cellular states characteristic of lymphoid tissue in different lymphomas and/or associated with different prognoses are included. Different cellular states characteristic of lymphoid tissue at different developmental stages are also included. By comparing expression profiles of different states, particularly a normal state and a lymphoma state, genes demonstrating altered expression in an oncogenic state, namely lymphoma, are determined. The difference in gene expression between the two states serves as the basis for lymphoma diagnosis, in which a tissue sample from a patient is analyzed to generate a gene expression profile which is compared to the normal and lymphoma expression profile to see which it more closely resembles. Further and similarly provided are means for assessing the developmental stage of lymphomas so recognized, by comparing the expression profile of a lymphoma sample to the expression profile of normal or diseased lymphoid tissue at various stages of development. Further and similarly provided are methods for determining the efficacy of lymphoma treatment, which comprise analyzing a tissue sample from a patient to generate a gene expression profile which is compared to the normal and lymphoma expression profile to see which it more closely resembles.

“Differential expression,” or grammatical equivalents as used herein, refers to both qualitative as well as quantitative differences in the genes' temporal and/or cellular expression patterns within and among the cells. Thus, a differentially expressed gene can qualitatively have its expression altered, including an activation or inactivation, in, for example, normal versus lymphoma tissue. That is, genes may be turned on or turned off in a particular state, relative to another state. As is apparent to the skilled artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e. upregulation or downregulation) is at least about 50%, more preferably at least about 100%, more preferably at least about 150%, more preferably, at least about 200%, with from 300 to at least 1000% being especially preferred.

As will be appreciated by those in the art, gene expression changes may be evaluated at both the gene transcript and protein level; that is, the amount of gene expression may be monitored using nucleic acid probes to the DNA or RNA equivalent of the gene transcript, or, alternatively, the final gene product itself (protein) can be monitored, for example through the use of antibodies to the Nrf2 protein and standard immunoassays (ELISAs, etc.) or other techniques, including mass spectroscopy assays, 2D gel electrophoresis assays, etc. Thus, the proteins corresponding to Nrf2 genes, i.e. those identified as being important in a lymphoma phenotype, can be evaluated in a lymphoma diagnostic test.

In a preferred embodiment, gene expression monitoring is done and a number of genes, i.e. an expression profile, is monitored simultaneously, although multiple protein expression monitoring can be done as well. Similarly, these assays may be done on an individual basis as well.

In this embodiment, the Nrf2 nucleic acid probes may be attached to biochips as outlined herein for the detection and quantification of Nrf2 sequences in a particular cell. The assays are done as is known in the art. As will be appreciated by those in the art, any number of different LA sequences may be used as probes, with single sequence assays being used in some cases, and a plurality of the sequences described herein being used in other embodiments. In addition, while solid-phase assays are described, any number of solution based assays may be done as well.

In a preferred embodiment, both solid and solution based assays may be used to detect Nrf2 sequences that are up-regulated or down-regulated in lymphoma as compared to normal lymphoid tissue. In instances where the Nrf2 sequence has been altered but shows the same expression profile or an altered expression profile, the protein will be detected as outlined herein.

In a preferred embodiment nucleic acids encoding the Nrf2 protein are detected. Although DNA or RNA encoding the Nrf2 protein may be detected, of particular interest are methods wherein the mRNA encoding a Nrf2 protein is detected. The presence of mRNA in a sample is an indication that the Nrf2 gene has been transcribed to form the mRNA, and suggests that the protein is being synthesized. Probes to detect the mRNA can be any nucleotide/deoxynucleotide probe that is complementary to and base pairs with the mRNA and includes but is not limited to oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a Nrf2 protein is detected by binding the digoxygenin with an anti-digoxygenin secondary antibody and developed with nitro blue tetrazolium and 5-bromo-4-chloro-3-indoyl phosphate.

The isolation of mRNA comprises isolating total cellular RNA by disrupting a cell and performing differential centrifugation. Once the total RNA is isolated, mRNA is isolated by making use of the adenine nucleotide residues known to those skilled in the art as a poly (A) tail found on virtually every eukaryotic mRNA molecule at the 3′ end thereof. Oligonucleotides composed of only deoxythymidine [olgo(dT)] are linked to cellulose and the oligo(dT)-cellulose packed into small columns. When a preparation of total cellular RNA is passed through such a column, the mRNA molecules bind to the oligo(dT) by the poly (A) tails while the rest of the RNA flows through the column. The bound mRNAs are then eluted from the column and collected.

In a preferred embodiment, any of the three classes of proteins as described herein (secreted, transmembrane or intracellular proteins) are used in diagnostic assays. The Nrf2 proteins, antibodies, nucleic acids, modified proteins and cells containing Nrf2 sequences are used in diagnostic assays. These assays can be directed to individual genes or corresponding polypeptides or to sets of genes or corresponding polypeptides.

As described and defined herein, Nrf2 proteins find use as markers of lymphoma. Detection of these proteins in putative lymphomic tissue or patients allows for a determination or diagnosis of lymphoma. Numerous methods known to those of ordinary skill in the art find use in detecting lymphoma. In one embodiment, antibodies are used to detect Nrf2 proteins. A preferred method separates proteins from a sample or patient by electrophoresis on a gel (typically a denaturing and reducing protein gel, but may be any other type of gel including isoelectric focusing gels and the like). Following separation of proteins, the Nrf2 protein is detected by immunoblotting with antibodies raised against the Nrf2 protein or a portion thereof. Methods of immunoblotting are well known to those of ordinary skill in the art.

In another preferred method, antibodies to the Nrf2 protein find use in in situ imaging techniques. In this method cells are contacted with from one to many antibodies to the Nrf2 protein(s). Following washing to remove non-specific antibody binding, the presence of the antibody or antibodies is detected. In one embodiment the antibody is detected by incubating with a secondary antibody that contains a detectable label. In another method the primary antibody to the Nrf2 protein(s) contains a detectable label. In another preferred embodiment each one of multiple primary antibodies contains a distinct and detectable label. This method finds particular use in simultaneous screening for a plurality of Nrf2 proteins. As will be appreciated by one of ordinary skill in the art, numerous other histological imaging techniques are useful in the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing lymphoma from blood samples. As previously described, certain Nrf2 proteins are secreted/circulating molecules. Blood samples, therefore, are useful as samples to be probed or tested for the presence of secreted Nrf2 proteins. Antibodies can be used to detect the Nrf2 by any of the previously described immunoassay techniques including ELISA, immunoblotting (Western blotting), immunoprecipitation, BIACORE technology and the like, as will be appreciated by one of ordinary skill in the art.

In a preferred embodiment, in situ hybridization of labeled Nrf2 nucleic acid probes to tissue arrays is done. For example, arrays of tissue samples, including Nrf2 tissue and/or normal tissue, are made. In situ hybridization as is known in the art can then be done.

It is understood that when comparing the expression fingerprints between an individual and a standard, the skilled artisan can make a diagnosis as well as a prognosis. It is further understood that the genes which indicate the diagnosis may differ from those which indicate the prognosis. It is further understood that genes concerned in prognosis may be expressed at levels similar to those of non-lymphoma lymphoid tissue, but serve in prognosis by association with lymphoma diagnosis.

In a preferred embodiment, the Nrf2 proteins, antibodies, nucleic acids, modified proteins and cells containing Nrf2 sequences are used in prognosis assays. As above, gene expression profiles can be generated that correlate to lymphoma severity, in terms of long term prognosis. Again, this may be done on either a protein or nucleic acid level, with the use of nucleotides being preferred. As above, the Nrf2 probes are attached to biochips for the detection and quantification of Nrf2 sequences in a tissue or patient sample. The assays proceed as outlined for diagnosis.

In a preferred embodiment, any of the Nrf2 sequences as described herein are used in drug screening assays. In one aspect, the Nrf2 proteins, antibodies, nucleic acids, modified proteins and cells containing Nrf2 sequences are used in drug screening assays to evaluate the effect of drug candidates on a “gene expression profile” or expression profile of polypeptides. In one embodiment, the expression profiles are used, preferably in conjunction with high throughput screening techniques to allow monitoring for expression profile genes after treatment with a candidate agent, Zlokarnik, et al., Science 279, 84-8 (1998), Heid, et al., Genome Res., 6:986-994 (1996).

In a preferred embodiment, the Nrf2 proteins, antibodies, nucleic acids, modified proteins and cells containing the native or modified Nrf2 proteins are used in screening assays. That is, the present invention provides novel methods for screening for compositions which modulate the lymphoma phenotype. As above, this can be done by screening for modulators of gene expression or for modulators of protein activity. Gene expression and protein activity may be evaluated on an individual gene and protein basis, or by evaluating the effect of drug candidates on a gene expression or protein expression profile. In a preferred embodiment, the expression profiles are used, preferably in conjunction with high throughput screening techniques to allow monitoring for expression profile genes after treatment with a candidate agent, see Zlokarnik, supra.

Having identified the Nrf2 genes herein, a variety of assays to evaluate the effects of agents on gene expression may be executed. In a preferred embodiment, assays may be run on an individual gene or protein level. That is, having identified a particular gene as aberrantly regulated in lymphoma, candidate bioactive agents may be screened to modulate the gene's response. “Modulation” thus includes both an increase and a decrease in gene expression or activity. The preferred amount of modulation will depend on the original change of the gene expression in normal versus tumor tissue, with changes of at least 10%, preferably 50%, more preferably 100-300%, and in some embodiments 300-1000% or greater. Thus, if a gene exhibits a 4 fold increase in tumor compared to normal tissue, a decrease of about four fold is desired; a 10 fold decrease in tumor compared to normal tissue makes a 10 fold increase in expression for a candidate agent desirable, etc. Alternatively, where the Nrf2 sequence has been altered but shows the same expression profile or an altered expression profile, the protein will be detected as outlined herein.

As will be appreciated by those in the art, this may be done by evaluation at either the gene transcript or the protein level; that is, the amount of gene expression may be monitored using nucleic acid probes and the quantification of gene expression levels, or, alternatively, the level of the gene product itself can be monitored, for example through the use of antibodies to the Nrf2 protein and standard immunoassays. Alternatively, binding and bioactivity assays with the protein may be done as outlined below.

In a preferred embodiment, gene expression monitoring is done and a number of genes, i.e. an expression profile, are monitored simultaneously, although multiple protein expression monitoring can be done as well.

In this embodiment, the Nrf2 nucleic acid probes are attached to biochips as outlined herein for the detection and quantification of Nrf2 sequences in a particular cell. The assays are further described below.

Generally, in a preferred embodiment, a candidate bioactive agent is added to the cells prior to analysis. Moreover, screens are provided to identify a candidate bioactive agent which modulates lymphoma, modulates Nrf2 proteins, binds to an Nrf2 protein, or interferes with the binding of an Nrf2 protein to another molecule. In a preferred embodiment, the interference is as the interference between Nrf2 protein binding to an anti-Nrf2 antibody. In another preferred embodiment, the interference is as the interference between Nrf2 protein binding to DNA. In one embodiment, such DNA comprises the Nrf2 binding sequence GCTGAGTCATGATGAGTCA. In one embodiment, the interference is as the interference between a portion of an Nrf2 protein binding to DNA. In one embodiment, such a portion of Nrf2 protein comprises a basic leucine zipper motif. In one embodiment, such a portion of Nrf2 protein comprises a basic leucine zipper motif comprising the amino acid sequence set forth by amino acids 474 to 518 in SEQ ID NO:2 and at Genbank accession number AAA68291. In one embodiment, such portion of Nrf2 protein comprises a basic leucine zipper motif comprising an amino acid sequence having at least about 90% identity to the amino acid sequence set forth by amino acids 474 to 518 in SEQ ID NO:2 and at Genbank accession number AAA68291. In one embodiment, such a portion of Nrf2 protein comprises a basic leucine zipper motif comprising the amino acid sequence set forth by amino acids 482 to 525, more preferably 482 to 504, in SEQ ID NO:4 and at Genbank accession number NP_{—}006155. In one embodiment, such a portion of Nrf2 protein comprises a basic leucine zipper motif comprising an amino acid sequence having at least about 90% identity to the amino acid sequence set forth by amino acids 482 to 525, more preferably 482 to 504, in SEQ ID NO:4 and at Genbank accession number NP_{—}006155.

The term “candidate bioactive agent” or “drug candidate” or grammatical equivalents as used herein describes any molecule, e.g., protein, oligopeptide, small organic or inorganic molecule, polysaccharide, polynucleotide, etc., to be tested for bioactivity characterized by an ability to directly or indirectly altering either the lymphoma phenotype, binding to and/or modulating the bioactivity of an Nrf2 protein, or alter the expression of a Nrf2 sequence, including both nucleic acid sequences and amino acid sequences. In a particularly preferred embodiment, the candidate agent suppresses a Nrf2 phenotype, for example causing an otherwise Nrf2 gene expression profile to demonstrate a normal lymphoid tissue gene expression profile. Similarly, the candidate agent preferably suppresses a severe Nrf2 phenotype. Generally a plurality of assay mixtures are run in parallel with different agent concentrations to obtain a differential response to the various concentrations. Typically, one of these concentrations serves as a negative control, i.e., at zero concentration or below the level of detection.

In one aspect, a candidate agent will neutralize the effect of an Nrf2 protein. By “neutralize” is meant that the activity of a protein is either inhibited or opposed so as to have substantially no effect on a cell.

Candidate agents encompass numerous chemical classes, though typically they are organic or inorganic molecules, preferably small organic compounds having a molecular weight of more than 100 and less than about 2,500 daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the methods of the invention. Particularly preferred in this embodiment are libraries of bacterial, fungal, viral, and mammalian proteins, with the latter being preferred, and human proteins being especially preferred.

In a preferred embodiment, the candidate bioactive agents are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

In a preferred embodiment, the candidate bioactive agents are nucleic acids, as defined above.

As described above generally for proteins, nucleic acid candidate bioactive agents may be naturally occurring nucleic acids, random nucleic acids, or “biased” random nucleic acids. For example, digests of procaryotic or eucaryotic genomes may be used as is outlined above for proteins.

In a preferred embodiment, the candidate bioactive agents are organic chemical moieties, a wide variety of which are available in the literature.

In assays for altering the expression profile of one or more Nrf2 genes, after the candidate agent has been added and the cells allowed to incubate for some period of time, the sample containing the target sequences to be analyzed is added to the biochip. If required, the target sequence is prepared using known techniques. For example, the sample may be treated to lyse the cells, using known lysis buffers, electroporation, etc., with purification and/or amplification such as PCR occurring as needed, as will be appreciated by those in the art. For example, an in vitro transcription with labels covalently attached to the nucleosides is done. Generally, the nucleic acids are labeled with a label as defined herein, with biotin-FITC or PE, cy3 and cy5 being particularly preferred.

In a preferred embodiment, the target sequence is labeled with, for example, a fluorescent, chemiluminescent, chemical, or radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. As known in the art, unbound labeled streptavidin is removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents may be included in the assays. These include reagents like salts, buffers, neutral proteins, e.g. albumin, detergents, etc which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target. In addition, either solid phase or solution based (i.e., kinetic PCR) assays may be used.

Once the assay is run, the data is analyzed to determine the expression levels, and changes in expression levels as between states, of individual genes, forming a gene expression profile.

In a preferred embodiment, as for the diagnosis and prognosis applications, having identified the differentially expressed gene(s) or mutated gene(s) important in any one state, screens can be run to alter the expression of the genes individually. That is, screening for modulation of regulation of expression of a single gene can be done. Thus, for example, particularly in the case of target genes whose presence or absence is unique between two states, screening is done for modulators of the target gene expression.

In addition, screens can be done for novel genes that are induced in response to a candidate agent. After identifying a candidate agent based upon its ability to suppress a lymphoma-associated expression pattern leading to a normal expression pattern, or modulate the expression of a single lymphoma-associated gene so that its expression in lymphoma-associated tissue mimics its expression in normal tissue, a screen as described above can be performed to identify genes that are specifically modulated in response to the agent. Comparing expression profiles between normal tissue, lymphoma-associated tissue, and agent treated lymphoma-associated tissue reveals genes that show a difference in expression in agent treated tissue as compared to both normal tissue and lymphoma-associated tissue. These agent specific sequences can be identified and used by any of the methods described herein for LA genes or proteins. In particular these sequences and the proteins they encode find use in marking or identifying agent treated cells. In addition, antibodies can be raised against the agent induced proteins and used to target novel therapeutics to the treated lymphoma-associated tissue sample.

Thus, in one embodiment, a candidate agent is administered to a population of lymphoma-associated cells, that thus has a lymphoma-associated gene expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, the cells can be washed if desired and are allowed to incubate under preferably physiological conditions for some period of time. The cells are then harvested and a new gene expression profile is generated, as outlined herein.

Thus, for example, lymphoma-associated tissue may be screened for agents that reduce or suppress the lymphoma-associated phenotype. A change in at least one gene of the expression profile indicates that the agent may have an effect on LA gene activity, more preferably Nrf2 gene activity.

By defining a signature gene expression profile for the lymphoma-associated phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual nucleic acids and gene products (proteins). That is, having identified a particular differentially expressed gene as important in a particular state, screening for modulators of either the corresponding mRNA or the corresponding protein can be done. In a preferred embodiment, the gene products of differentially expressed genes are sometimes referred to herein as Nrf2 proteins. An Nrf2 protein may be a fragment, or alternatively, may be the full length protein comprising the fragment encoded by the nucleic acid sequences set forth in SEQ ID NO:1 or SEQ ID NO:3. Preferably, the Nrf2 protein is a fragment. In another embodiment, Nrf2 proteins comprise amino acid sequence variants as further described herein.

Preferably, the Nrf2 protein is a fragment of approximately 14 to 24 amino acids long. More preferably the fragment is a soluble fragment. Preferably, the fragment includes a non-transmembrane region. In a preferred embodiment, the fragment has an N-terminal Cys to aid in solubility. In one embodiment, the c-terminus of the fragment is kept as a free acid and the n-terminus is a free amine to aid in coupling, i.e., to cysteine.

In one embodiment the Nrf2 proteins are conjugated to an immunogenic agent as discussed herein. In one embodiment the Nrf2 protein is conjugated to BSA.

In a preferred embodiment, screens for agents that alter the biological function of the expression product of the Nrf2 gene are done. Again, having identified the importance of a gene in a particular state, screening for agents that bind and/or modulate the biological activity of the gene product can be run as is more fully outlined below.

In a preferred embodiment, screens are designed to first find candidate agents that can bind to Nrf2 proteins, and then these agents may be used in assays that evaluate the ability of the candidate agent to modulate the Nrf2 activity and the lymphoma phenotype. As will be appreciated by those in the art, there are a number of different assays which may be run to assess binding and activity.

In a preferred embodiment, binding assays are done. In general, purified or isolated gene product is used; that is, the gene products of one or more Nrf2 nucleic acids are made. In general, this is done as is known in the art. For example, antibodies are generated to the protein gene products for use in the purification of proteins, as well as for use in standard immunoassays run to determine the amount of protein present. Alternatively, cells comprising the Nrf2 proteins can be used in the assays.

In a preferred embodiment, the methods comprise combining a Nrf2 protein and a candidate bioactive agent, and determining the binding of the candidate agent to the Nrf2 protein. Preferred embodiments utilize the human or mouse Nrf2 protein, although other mammalian proteins may also be used, for example for the development or assessment of animal models of human disease. In some embodiments, as outlined herein, variant or derivative Nrf2 proteins may be used.

Generally, in a preferred embodiment of the methods herein, the Nrf2 protein or the candidate agent is non-diffusably bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the Nrf2 protein is bound to the support, and a candidate bioactive agent is added to the assay. Alternatively, the candidate agent is bound to the support and the Nrf2 protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the Nrf2 protein may be done in a number of ways. In a preferred embodiment, the candidate bioactive agent is labeled, and binding determined directly. For example, this may be done by attaching all or a portion of the Nrf2 protein to a solid support, adding a labeled candidate agent (for example a fluorescent label), washing off excess reagent, and determining whether the label is present on the solid support. Various blocking and washing steps may be utilized as is known in the art.

By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the proteins (or proteinaceous candidate agents) may be labeled at tyrosine positions using ¹²⁵I, or with fluorophores. Alternatively, more than one component may be labeled with different labels; using ¹²⁵I for the proteins, for example, and a fluorophor for the candidate agents.

In a preferred embodiment, the binding of the candidate bioactive agent is determined through the use of competitive binding assays. In this embodiment, the competitor is a binding moiety known to bind to the target molecule (i.e. Nrf2 protein), such as an antibody, peptide, binding partner, ligand, etc. Under certain circumstances, there may be competitive binding as between the bioactive agent and the binding moiety, with the binding moiety displacing the bioactive agent.

In a preferred embodiment, the Nrf2 binding moiety is a nucleic acid comprising the Nrf2 binding sequence GCTGAGTCATGATGAGTCA. In another preferred embodiment, the Nrf2 binding moiety is a transcriptional cofactor involved in Nrf2-mediated gene regulation. In a preferred embodiment, the DNA binding domain of Nrf2 is used in binding assays. In one embodiment, the transcriptional activation domain of Nrf2 is used in binding assays.

In one embodiment, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate bioactive agent. Displacement of the competitor is an indication that the candidate bioactive agent is binding to the Nrf2 protein and thus is capable of binding to, and potentially modulating, the activity of the Nrf2 protein. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the Nrf2 protein with a higher affinity. Thus, if the candidate bioactive agent is labeled, the presence of the label on the support, coupled with a lack of competitor binding, may indicate that the candidate agent is capable of binding to the Nrf2 protein.

In a preferred embodiment, the methods comprise differential screening to identity bioactive agents that are capable of modulating the activity of the Nrf2 proteins. In this embodiment, the methods comprise combining a Nrf2 protein and a competitor in a first sample. A second sample comprises a candidate bioactive agent, a Nrf2 protein and a competitor. The binding of the competitor is determined for both samples, and a change, or difference in binding between the two samples indicates the presence of an agent capable of binding to the Nrf2 protein and potentially modulating its activity. That is, if the binding of the competitor is different in the second sample relative to the first sample, the agent is capable of binding to the Nrf2 protein.

Alternatively, a preferred embodiment utilizes differential screening to identify drug candidates that bind to the native Nrf2 protein, but cannot bind to modified Nrf2 proteins. The structure of the Nrf2 protein may be modeled, and used in rational drug design to synthesize agents that interact with putative binding sites within. Drug candidates that affect Nrf2 bioactivity are also identified by screening drugs for the ability to either enhance or reduce the activity of the protein.

In a preferred embodiment, transcription assays as known in the art, for example as disclosed in (Ausubel, supra) and Caterina et al., NAR 22:2383-2391, 1994, are used in screens to identify candidate bioactive agents that can affect Nrf2 protein activity, particularly transcription regulating activity. In a preferred embodiment, the transcription assays employ the Nrf2 DNA binding sequence GCTGAGTCATGATGAGTCA. In a preferred embodiment, an Nrf2 protein comprises the amino acid sequence st forth in SEQ ID NO:2 and at Genbank accession number AAA68291, or a fragment thereof. In another preferred embodiment, an Nrf2 protein comprises the amino acid sequence set forth in SEQ ID NO:4 and at Genbank accession number NP_{—}006155, or a fragment thereof. In another preferred embodiment, an Nrf2 protein comprises the amino acid sequence set forth by amino acids 477 to 518 in SEQ ID NO:2 and at Genbank accession number AAA68291. In another preferred embodiment, an Nrf2 protein comprises the amino acid sequence set forth by amino acids 482 to 526, more preferably 482 to 504, in SEQ ID NO:4 and at Genbank accession number NP_{—}006155.

In one embodiment, the portion of Nrf2 protein used comprises the DNA binding domain, such as the basic domain of a basic leucine zipper domain-containing protein. In one embodiment, the portion of Nrf2 used comprises the transcriptional activation domain, such as the acidic domain of a basic leucine zipper domain-containing protein.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

Screening for agents that modulate the activity of Nrf2 proteins may also be done. In a preferred embodiment, methods for screening for a bioactive agent capable of modulating the activity of Nrf2 proteins comprise the steps of adding a candidate bioactive agent to a sample of Nrf2 proteins, as above, and determining an alteration in the biological activity of Nrf2 proteins. “Modulating the activity of an Nrf2 protein” includes an increase in activity, a decrease in activity, or a change in the type or kind of activity present. Thus, in this embodiment, the candidate agent may bind to Nrf2 proteins (although this may not be necessary), and should alter its biological or biochemical activity as defined herein. The methods include both in vitro screening methods, as are generally outlined above, and in vivo screening of cells for alterations in the presence, distribution, activity or amount of Nrf2 proteins.

Thus, in this embodiment, the methods comprise combining a Nrf2 sample and a candidate bioactive agent, and evaluating the effect on Nrf2 activity. By “LA activity” or grammatical equivalents herein is meant one of the Nrf2 protein's biological activities, including, but not limited to, its role in lymphoma, including cell division, preferably in lymphoid tissue, cell proliferation, tumor growth and transformation of cells. In one embodiment, Nrf2 activity includes activation of or by a protein encoded by a nucleic acid of the table. An inhibitor of Nrf2 activity is the inhibition of any one or more Nrf2 activities.

In a preferred embodiment, the activity of the Nrf2 protein is increased; in another preferred embodiment, the activity of the Nrf2 protein is decreased. Thus, bioactive agents that are antagonists are preferred in some embodiments, and bioactive agents that are agonists may be preferred in other embodiments.

In a preferred embodiment, the invention provides methods for screening for bioactive agents capable of modulating the activity of a Nrf2 protein. The methods comprise adding a candidate bioactive agent, as defined above, to a cell comprising Nrf2 proteins. Preferred cell types include almost any cell. The cells contain a recombinant nucleic acid that encodes a Nrf2 protein. In a preferred embodiment, a library of candidate agents are tested on a plurality of cells.

In one aspect, the assays are done in the presence or absence of physiological signals such as but not limited to hormones, antibodies, peptides, antigens, cytokines, growth factors, action potentials, pharmacological agents including chemotherapeutics, radiation, carcinogens, or other cells (i.e. cell-cell contacts). In one aspect, the assays are done prior to or following exposure to physiological signals such as but not limited to hormones, antibodies, peptides, antigens, cytokines, growth factors, action potentials, pharmacological agents including chemotherapeutics, radiation, carcinogens, or other cells (i.e. cell-cell contacts). In another example, the determinations are determined at different stages of the cell cycle process.

In this way, bioactive agents are identified. Compounds with pharmacological activity are able to enhance or interfere with the activity of the Nrf2 protein.

In one embodiment, a method of inhibiting lymphoma cancer cell division is provided. The method comprises administration of a lymphoma cancer inhibitor. In a preferred embodiment, such a method comprises administration of an Nrf2 inhibitor.

In another embodiment, a method of inhibiting tumor growth is provided. The method comprises administration of a lymphoma cancer inhibitor. In a preferred embodiment, such a method comprises administration of an Nrf2 inhibitor.

In a further embodiment, methods of treating cells or individuals with cancer are provided. The method comprises administration of a lymphoma cancer inhibitor. In a preferred embodiment, such a method comprises administration of an Nrf2 inhibitor.

In one embodiment, a lymphoma cancer inhibitor is an antibody as discussed above. In another embodiment, the lymphoma cancer inhibitor is an antisense molecule. Antisense molecules as used herein include antisense or sense oligonucleotides comprising a singe-stranded nucleic acid sequence (either RNA or DNA) capable of binding to target mRNA (sense) or DNA (antisense) sequences for lymphoma cancer molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen, Cancer Res. 48:2659, (1988) and van der Krol et al., BioTechniques 6:958, (1988).

Antisense molecules may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide may be introduced into a cell containing the target nucleic acid sequence by formation of an oligonucleotide-lipid complex, as described in WO 90/10448. It is understood that the use of antisense molecules or knock out and knock in models may also be used in screening assays as discussed above, in addition to methods of treatment.

The compounds having the desired pharmacological activity may be administered in a physiologically acceptable carrier to a host, as previously described. The agents may be administered in a variety of ways, orally, parenterally e.g., subcutaneously, intraperitoneally, intravascularly, etc. Depending upon the manner of introduction, the compounds may be formulated in a variety of ways. The concentration of therapeutically active compound in the formulation may vary from about 0.1-100% wgt/vol. The agents may be administered alone or in combination with other treatments, i.e., radiation.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Without being bound by theory, it appears that the various Nrf2 sequences are important in lymphoma. Accordingly, disorders based on mutant or variant Nrf2 genes may be determined. In one embodiment, the invention provides methods for identifying cells containing variant Nrf2 genes comprising determining all or part of the sequence of at least one endogenous Nrf2 genes in a cell. As will be appreciated by those in the art, this may be done using any number of sequencing techniques. In a preferred embodiment, the invention provides methods of identifying the Nrf2 genotype of an individual comprising determining all or part of the sequence of at least one Nrf2 gene of the individual. This is generally done in at least one tissue of the individual, and may include the evaluation of a number of tissues or different samples of the same tissue. The method may include comparing the sequence of the sequenced Nrf2 gene to a known Nrf2 gene, i.e., a wild-type gene. As will be appreciated by those in the art, alterations in the sequence of some oncogenes can be an indication of either the presence of the disease, or propensity to develop the disease, or prognosis evaluations.

The sequence of all or part of the Nrf2 gene can then be compared to the sequence of a known Nrf2 gene to determine if any differences exist. This can be done using any number of known homology programs, such as Bestfit, etc. In a preferred embodiment, the presence of a difference in the sequence between the Nrf2 gene of the patient and the known Nrf2 gene is indicative of a disease state or a propensity for a disease state, as outlined herein.

It will be recognized that in some cases, particularly those concerning tumor suppresser genes, or recessive mutations generally, Nrf2 sequences characteristic of an Nrf2 phenotype will be found in normal lymphoid tissue. In these case it will be recognized that other Nrf2 gene alleles found in the tissue are likely involved in the maintenance of the normal lymphoid phenotype.

It will also be recognized that many transcription factors function as multimers, and as such, dominant negative effects in respect of the physiological processes they regulate are often encountered with altered alleles. That is, a single alternate allele (alternate in respect of the recognized wildtype allele) is often sufficient to alter transcription as normally regulated by wildtype protein, through protein-protein interactions and the dominant dysfunction of an alternate protein.

In a preferred embodiment, the Nrf2 genes are used as probes to determine the number of copies of the Nrf2 gene in the genome. For example, some cancers exhibit chromosomal deletions or multiplications, resulting in an alteration in the copy number of a gene.

In another preferred embodiment Nrf2 genes are used as probes to determine the chromosomal location of the Nrf2 genes. Information such as chromosomal location finds use in providing a diagnosis or prognosis in particular when chromosomal abnormalities such as translocations, and the like are identified in Nrf2 gene loci.

Thus, in one embodiment, methods of modulating Nrf2 in cells or organisms are provided. In one embodiment, the methods comprise administering to a cell an anti-LA antibody that reduces or eliminates the biological activity of an endogenous Nrf2 protein. Alternatively, the methods comprise administering to a cell or organism a recombinant nucleic acid encoding a Nrf2 protein. As will be appreciated by those in the art, this may be accomplished in a number of ways. In a preferred embodiment, for example when the Nrf2 gene is down-regulated in lymphoma, the activity of the Nrf2 gene is increased by increasing the amount of Nrf2 sequence in the cell, for example by overexpressing the endogenous Nrf2 or by administering the Nrf2 nucleic acid sequence, using known gene-therapy techniques, for example. In a preferred embodiment, the gene therapy techniques include the incorporation of the exogenous gene using enhanced homologous recombination (EHR), for example as described in PCT/US93/03868, hereby incorporated by reference in its entirety. Alternatively, for example when the Nrf2 sequence is up-regulated in lymphoma, the activity of the endogenous Nrf2 gene is decreased, for example by the administration of a Nrf2 antisense nucleic acid.

In one embodiment, the Nrf2 proteins of the present invention may be used to generate polyclonal and monoclonal antibodies to Nrf2 proteins, which are useful as described herein. Similarly, the Nrf2 proteins can be coupled, using standard technology, to affinity chromatography columns. These columns may then be used to purify Nrf2 antibodies. In a preferred embodiment, the antibodies are generated to epitopes unique to a Nrf2 protein; that is, the antibodies show little or no cross-reactivity to other proteins. These antibodies find use in a number of applications. For example, the Nrf2 antibodies may be coupled to standard affinity chromatography columns and used to purify Nrf2 proteins. The antibodies may also be used as blocking polypeptides, as outlined above, since they will specifically bind to the Nrf2 protein.

In one embodiment, a therapeutically effective dose of a Nrf2 protein or modulator thereof is administered to a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for Nrf2 degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The administration of the Nrf2 proteins and modulators of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the Nrf2 proteins and modulators may be directly applied as a solution or spray.

The pharmaceutical compositions of the present invention comprise a Nrf2 protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

In a preferred embodiment, Nrf2 proteins and modulators are administered as therapeutic agents, and can be formulated as outlined above. Similarly, Nrf2 genes (including both the full-length sequence, partial sequences, or regulatory sequences of the Nrf2 coding regions) can be administered in gene therapy applications, as is known in the art. These Nrf2 genes can include antisense applications, either as gene therapy (i.e. for incorporation into the genome) or as antisense compositions, as will be appreciated by those in the art.

In a preferred embodiment, Nrf2 genes are administered as DNA vaccines, either single genes or combinations of Nrf2 genes. Naked DNA vaccines are generally known in the art as in Brower, Nature Biotechnology, 16:1304-1305 (1998), herein incorporated in its entirety by reference.

In one embodiment, Nrf2 genes of the present invention are used as DNA vaccines. Methods for the use of genes as DNA vaccines are well known to one of ordinary skill in the art, and include placing a Nrf2 gene or portion of a Nrf2 gene under the control of a promoter for expression in a Nrf2 patient. The Nrf2 gene used for DNA vaccines can encode full-length Nrf2 proteins, but more preferably encodes portions of the Nrf2 proteins including peptides derived from the Nrf2 protein. In a preferred embodiment a patient is immunized with a DNA vaccine comprising a plurality of nucleotide sequences derived from a Nrf2 gene. Similarly, it is possible to immunize a patient with a plurality of Nrf2 genes or portions thereof as defined herein. Without being bound by theory, expression of the polypeptide encoded by the DNA vaccine, cytotoxic T-cells, helper T-cells and antibodies are induced which recognize and destroy or eliminate cells expressing Nrf2 proteins.

In a preferred embodiment, the DNA vaccines include a gene encoding an adjuvant molecule with the DNA vaccine. Such adjuvant molecules include cytokines that increase the immunogenic response to the Nrf2 polypeptide encoded by the DNA vaccine. Additional or alternative adjuvants are known to those of ordinary skill in the art and find use in the invention.

In another preferred embodiment Nrf2 genes find use in generating animal models of lymphoma. As is appreciated by one of ordinary skill in the art, when the Nrf2 gene identified is repressed or diminished in Nrf2 tissue, gene therapy technology wherein antisense RNA is directed to the Nrf2 gene will also diminish or repress expression of the gene. An animal generated as such serves as an animal model of Nrf2 that finds use in screening bioactive drug candidates. Similarly, gene knockout technology including gene disruption, for example as a result of homologous recombination with an appropriate gene targeting vector, will result in the absence of the Nrf2 protein as encoded by the targeted allele. When desired, tissue-specific expression or knockout of the Nrf2 gene may be necessary.

It is also possible that the Nrf2 protein is overexpressed in lymphoma. As such, transgenic animals can be generated that overexpress the Nrf2 protein. Depending on the desired expression level, promoters of various strengths can be employed to express the transgene. Also, the number of copies of the integrated transgene can be determined; as will be appreciated by those in the art, transgene expression level is often different between animals having different numbers of copies of a transgene. Animals generated by such methods find use as animal models of lymphoma, and more generally for the study of dysregulated growth, survival, or apoptosis in cells of the hematopoietic system, and are additionally useful in screening for bioactive molecules to treat lymphoma.

The Nrf2 nucleic acid sequences of the invention are depicted in Tables 1 through 4.

A Nrf2 nucleic acid sequence of the invention is depicted in Table 1 as SEQ ID NO. 1. The nucleic acid sequence shown is from mouse.

TABLE 1
MOUSE

| SEQ ID# | SEQUENCE |
|---|---|
| 1 | TGCTCCATGCCCTTGTCCTCGCTCTGGCCCTTGCCTCTTGCCCTAGCCTTTTCTCCGCCTCTAAGTTCTTGTCC |
| | CGTCCCTAGGTCCTTGTTCCAGGGGGTGGGGGCGGGGCGGACTAAGGCTGGCCTGCCACTCCAGCGAGCAG |
| | GCTATCTCCTAGTTCTCGCTGCTCGGACTAGCCATTGCCGCCGCCTCACCTCTGCTGCAAGTAGCCTCGCCGT |
| | CGGGGAGCCCTACCACACGGTCCGCCCTCAGCATGATGGACTTGGAGTTGCCACCGCCAGACTACAGTCCCA |
| | GCAGGACATGGATTTGATTGACATCCTTTGGAGGCAAGACATAGATCTTGGAGTAAGTCGAGAAGTGTTTGAC |
| | TTTAGTCAGCGACAGAAGGACTATGAGCTGGAAAAACAGAAAAAACTCGAAAAGGAAAGACAAGAGCAACTCC |
| | AGAAGGAACAGGAGAAGGCCTTTTTTGCTCAGTTTCAACTGGATGAAGAAACAGGAGAATTCCTCCCAATTCA |
| | GCCGGCCCAGCACATCCAGACAGACACCAGTGGATCCGCCAGCTACTCCCAGGTTGCCCACATTCCCAAACA |
| | AGATGCCTTGTACTTTGAAGACTGTATGCAGCTTTTGGCAGAGACATTCCCATTTGTAGATGACCATGAGTCGC |
| | TTGCCCTGGATATCCCCAGCCACGCTGAAAGTTCAGTCTTCACTGCCCCTCATCAGGCCCAGTCCCTCAATAG |
| | CTCTCTGGAGGCAGCCATGACTGATTTAAGCAGCATAGAGCAGGACATGGAGCAAGTTTGGCAGGAGCTATTT |
| | TCCATTCCCGAATTACAGTGTCTTAATACCGAAAACAAGCAGCTGGCTGATACTACCGCTGTTCCCAGCCCAGA |
| | AGCCACACTGACAGAAATGGACAGCAATTACCATTTTTACTCATCGATCTCCTCGCTGGAAAAAGAAGTGGGCA |
| | ACTGTGGTCCACATTTCCTTCATGGTTTTGAGGATTCTTTCAGCAGCATCCTCTCCACTGATGATGCCAGCCAG |
| | CTGACCTCCTTAGACTCAAATCCCACCTTAAACACAGATTTTGGCGATGAATTTTATTCTGCTTTCATAGCAGAG |
| | CCCAGTGACGGTGGCAGCATGCCTTCCTCCGCTGCCATCAGTCAGTCACTCTCTGAACTCCTGGACGGGACTA |
| | TTGAAGGCTGTGACCTGTCACTGTGTAAAGCTTTCAACCCGAAGCACGCTGAAGGCACAATGGAATTCAATGA |
| | CTCTGACTCTGGCATTTCACTGAACACGAGTCCCAGCCGAGCGTCCCCAGAGCACTCCGTGGAGTCTTCCATT |
| | TACGGAGACCCACCGCCTGGGTTCAGTGACTCGGAAATGGAGGAGCTAGATAGTGCCCCTGGAAGTGTCAAA |
| | CAGAACGGCCCTAAAGCACAGCCAGCACATTCTCCTGGAGACACAGTACAGCCTCTGTCACCAGCTCAAGGG |
| | CACAGTGCTCCTATGCGTGAATCCCAATGTGAAAATACAACAAAAAAAAACCCGTGAGTCCTGGTCATCA |
| | AAAAGCCCCATTCACAAAAGACAAACATTCAAGCCGCTTAGAGGCTCATCTCACACGAGATGAGCTTAGGGCA |
| | AAAGCTCTCCATATTCCATTCCCTGTCGAAAAAATCATTAACCTCCCTGTTGATGACTTCAATGAAATGATGTCC |
| | AAGAACAATTCAATGAAGCTCAGCTCGCATTGATCCGAGATATACGCAGGAGAGGTAAAGAATAAGTCGCCG |
| | CCCAGAACTGTAGGAAAAGGAAGCTGGAGAACATTGTCGAGCTGGAGCAAGACTTGGGCCACTTAAAAGACG |
| | AGAGAGCAATTCAATGAAGCTCAGCTCGCATTGATCCGAGATATACGCAGGAGAGGTAAGAATAAAGTCGCCG |
| | CTTGTATCTTGAAGTCTTCAGCATGTTACGTGATGAGGATGGAAAAAGCCTTACTCTCCCAGTGAATACTCTCTGC |
| | AGCAAACCAGAGATGGCAATGTGTTCCTTGTTCCCAAAAGCAAGAAGCCAGATACAAAGAAAAACTAGGTTCG |
| | GGAGGATGGAGCCTTTTCTGAGCTAGTGTTTGTTTTGTACTGCTAAAACTTCCTACTGTGATGTGAAATGCAGA |
| | AACACTTTATAAGTAACTATGCAGAATTATAGCCAAAGCTAGTATAGCAATAATATGAAACTTTACAAAGCATTA |
| | AAGTCTCAATGTTGAATCAGTTTCATTTTAACTCTCAAGTTAATTTCTTAGGCACCATTTGGGAGAGTTTCTGTTT |
| | AAGTGTAAATACTACAGAACTTATTTATACTGTTCTCACTTGTTACAGTCATAGACTTATATGACATCTGGCTAAA |
| | AGCAAACTATTGAAAACTAACCAGACCACTATACTTTTTTATATACTGTATGAACAGGAAATGACATTTTTATATT |
| | AAATTGTTTAGCTCATAAAAATTAAGGAGCTAGCACTAATAAAAGAATATCATGACT |

SEQ ID NO. 2 (in Table 2) represents the amino acid sequence of a protein encoded by SEQ ID NO. 1.

TABLE 2
MOUSE

| SEQ ID# | SEQUENCE |
|---|---|
| 2 | MDLIDILWRQDIDLGVSREVFDFSQRQKDYELEKQKKLEKERQEQLQKEQEKAFFAQFQLDEETGEFLPIQPAQHIQ |
| | TDTSGSASYSQVAHIPKQDALYFEDCMQLLAETFPFVDDHESLALDIPSHAESSVFTAPHQAQSLNSSLEAAMTDLS |
| | SIEQDMEQVWQELFSIPELQCLNTENKQLADTTAVPSPEATLTEMDSNYHFYSSISSLEKEVGNCGPHFLHGFEDS |
| | FSSILSTDDASQLTSLDSNPTLNTDFGDEFYSAFIAEPSDGGSMPSSAAISQSLSELLDGTIEGCDLSLCKAFNPKHA |
| | EGTMEFNDSDSGISLNTSPSRASPEHSVESSIYGDPPPGFSDSEMEELDSAPGSVKQNGPKAQPAHSPGDTVQPL |
| | SPAQGHSAPMRESQCENTTKKEVPVSPGHQKAPFTKDKHSSRLEAHLTRDELRAKALHIPFPVEKIINLPVDDFNE |
| | MMSKEQFNEAQLALIRDIRRRGKNKVAAQNCRKRKLENIVELEQDLGHLKDEREKLLREKGENDRNLHLLKRRLSTL |
| | YLEVFSMLRDEDGKPYSPSEYSLQQTRDGNVFLVPKSKKPDTKKN |

Table 3 (SEQ ID NO: 3) depicts a human Nrf2 nucleic acid sequence of the invention.

TABLE 3
HUMAN

| SEQ ID# | SEQUENCE |
|---|---|
| 3 | TTGGAGCTGCCGCCGCCGGGACTCCCGTCCCAGCAGGACATGGATTTGATTGACATACTTTGGAGGCAAGAT |
| | ATAGATCTTGGAGTAAGTCGAGAAGTATTTGACTTCAGTCAGCGACGGAAAGAGTATGAGCTGGAAAAACAGA |
| | AAAAACTTGAAAAGGAAAGACAAGAACAACTCCAAAAGGAGCAAGAGAAAGCCTTTTTCACTCAGTTACAACTA |
| | GATGAAGAGACAGGTGAATTTCTCCCAATTCAGCCAGCCCAGCACACCCAGTCAGAAACCAGTGGATCTGCCA |
| | ACTACTCCCAGGTTGCCCACATTCCCAAATCAGATGCTTTGTACTTTGATGACTGCATGCAGCTTTTGGCGCAG |
| | ACATTCCCGTTTGTAGATGACAATGAGGTTTCTTCGGCTACGTTTCAGTCACTTGTTCCTGATATTCCCGGTCA |
| | CATCGAGAGCCCAGTCTTCATTGCTACTAATCAGGCTCAGTCACCTGAAACTTCTGTTGCTCAGGTAGCCCCTG |
| | TTGATTTAGACGGTATGCAACAGGACATTGAGCAAGTTTGGGAGGAGCTATTATCCATTCCTGAGTTACAGTGT |
| | CTTAATATTGAAAATGACAAGCTGGTTGAGACTACCATGGTTCCAAGTCCAGAAGCCAAACTGACAGAAGTTGA |
| | CAATTATCATTTTTACTCATCTATACCCTCAATGGAAAAAGAAGTAGGTAACTGTAGTCCACATTTTCTTAATGCT |
| | TTTGAGGATTCCTTCAGCAGCATCCTCTCCACAGAAGACCCCAACCAGTTGACAGTGAACTCATTAAATTCAGA |
| | TGCCACAGTGAACACAGATTTTGGTGATGAATTTTATTGTGCTTTCATAGCTGAGCCCAGTATCAGCAACAGCA |
| | TGCCCTCACCTGCTACTTTAAGCCATTCACTCTCTGAACTTCTAAATGGGCCCATTGATGTTTCTGATCTATCAC |
| | TTTGCAAAGCTTTCAACCAAAACCACCCTGAAAGCACAGCAGAATTCAATGATTCTGACTCCGGCATTTCACTA |
| | AACACAAGTCCCAGTGTGGCATCACCAGAACACTCAGTGGAATCTTCCAGCTATGGAGACACACTACTTGGCC |
| | TCAGTGATTCTGAAGTGGAAGAGCTAGATAGTGCCCCTGGAAGTGTCAAACAGAATGGTCCTAAAACACCAGT |
| | ACATTCTTCTGGGGATATGGTACAACCCTTGTCACCATCTCAGGGGCAGAGCACTCACGTGCATGATGCCCAA |
| | TGTGAGAACACACCAGAGAAAGAATTGCCTGTAAGTCCTGGTCATCGGAAAACCCCATTCACAAAAGACAAAC |
| | ATTCAAGCCGCTTGGAGGCTCATCTCACAAGAGATGAACTTAGGGCAAAAGCTCTCCATATCCCATTCCCTGTA |
| | GAAAAAATCATTAACCTCCCTGTTGTTGACTTCAACGAAATGATGTCCAAAGAGCAGTTCAATGAAGCTCAACT |
| | TGCATTAATTCGGGATATACGTAGGAGGGGTAAGAATAAAGTGGCTGCTCAGAATTGCAGAAAAAGAAAACTG |
| | GAAAATATAGTAGAACTAGAGCAAGATTTAGATCATTTGAAAGATGAAAAAGAAAAATTGCTCAAAGAAAAAGG |
| | AGAAAATGACAAAAGCCTTCACCTACTGAAAAAACAACTCAGCACCTTATATCTCGAAGTTTTCAGCATGCTAC |
| | GTGATGAAGATGGAAAACCTTATTCTCCTAGTGAATACTCCCTGCAGCAAACAAGAGATGGCAATGTTTTCCTT |
| | GTTCCCAAAAGTAAGAAGCCAGATGTTAAGAAAAACTAGATTTAGGAGGATTTGACCTTTTCTGAGCTAGTTTTT |
| | TTGTACTATTATACTAAAAGCTCCTACTGTGATGTGAAATGCTCATACTTTATAAGTAATTCTATGCAAAATCATA |
| | GCCAAAACTAGTATAGAAAATAATACGAAACTTTAAAAAGCATTGGAGTGTCAGTATGTTGAATCAGTAGTTTCA |
| | CTTTAACTGTAAACAATTTCTTAGGACACCATTTGGGCTAGTTTCTGTGTAAGTGTAAATACTACAAAAACTTATT |
| | TATACTGTTCTTATGTCATTTGTTATATTCATAGATTATATGATGATATGACATCTGGCTAAAAAGAAATTATTG |
| | CAAAACTAACCACGATGTACTTTTTTATAAATACTGTATGGACAAAAAATGGCATTTTTTATAATTAAATTGTTTA |
| | GCTCTGGCAAAAAAAAAAAATTTTTTAAGAGCTGGTACTAATAAAGGATTATTATGACTGTTAAAAAAAAAAAAA |
| | AAAAA |

Table 4 (SEQ ID NO: 4 depicts the amino acid sequence encoded by the nucleic acid sequence of SEQ ID NO: 3).

TABLE 4
HUMAN

| SEQ ID# | SEQUENCE |
|---|---|
| 3 | MDLIDILWRQDIDLGVSREVFDFSQRRKEYELEKQKKLEKERQEQLQKEQEKAFFTQLQLDEETGEFLPIQPAQHTQ |
| | SETSGSANYSQVAHIPKSDALYFDDCMQLLAQTFPFVDDNEVSSATFQSLVPDIPGHIESPVFIATNQAQSPETSVA |
| | QVAPVDLDGMQQDIEQVWEELLSIPELQCLNIENDKLVETTMVPSPEAKLTEVDNYHFYSSIPSMEKEVGNCSPHFL |
| | NAFEDSFSSILSTEDPNQLTVNSLNSDATVNTDFGDEFYSAFIAEPSISNSMPSPATLSHSLSELLNGPIDVSDLSLCK |
| | AFNQNHPESTAEFNDSDSGISLNTSPSVASPEHSVESSSYGDTLLGLSDSEVEELDSAPGSVKQNGPKTPVHSSG |
| | DMVQPLSPSQGQSTHVHDAQCENTPEKELPVSPGHRKTPFTKDKHSSRLEAHLTRDELRAKAALHIPFPVEKIINLPV |
| | VDFNEMMSKEQFNEAQLALIRDIRRRGKNKVAAQNCRKRKLENIVELEQDLDHLKDEKEKLLKEKGENOKSLHLLKK |
| | QLSTLYLEVFSMLRDEDGKPYSPSEYSLQQTRDGNVFLVPKSKKPDVKKN |

All accession numbers cited herein are incorporated by reference in their entirety. All references cited herein are expressly incorporated in their entirety by reference.

We claim:

1. A recombinant nucleic acid comprising a nucleotide sequence selected from the group consisting of the nucleic acid sequences set forth in SEQ ID NO:1 and SEQ ID NO:3.

2. A host cell comprising the recombinant nucleic acid of claim 1.

3. An expression vector comprising the recombinant nucleic acid according to claim 2.

4. A host cell comprising the expression vector of claim 3.

5. A recombinant protein comprising an amino acid sequence selected from the group consisting of the amino acid sequences set forth in SEQ ID NO:2 and SEQ ID NO:4.

6. A method of screening drug candidates comprising:
a) providing a cell that expresses an Nrf2 gene selected from the group consisting of the nucleic acid sequences set forth in SEQ ID NO:1 and SEQ ID NO:3, or a fragment thereof;
b) adding a drug candidate to said cell; and
c) determining the effect of said drug candidate on the expression of said Nrf2 gene.

7. A method according to claim 6 wherein said determining comprises comparing the level of expression in the absence of said drug candidate to the level of expression in the presence of said drug candidate.

8. A method of screening for a bioactive agent capable of binding to an Nrf2 protein, wherein said Nrf2 is encoded by a nucleic acid selected from the group consisting of the nucleic acid sequences set forth in SEQ ID NO:1 and SEQ ID NO:3, said method comprising:
a) combining said Nrf2 protein and a candidate bioactive agent; and
b) determining the binding of said candidate agent to said Nrf2 protein.

9. A method for screening for a bioactive agent capable of modulating the activity of an Nrf2 protein, wherein said Nrf2 protein is encoded by a nucleic acid selected from the group consisting of the nucleic acid sequences set forth in SEQ ID NO:1 and SEQ ID NO:3, said method comprising:
a) combining said Nrf2 protein and a candidate bioactive agent; and
b) determining the effect of said candidate agent on the bioactivity of said Nrf2 protein.

10. A method of evaluating the effect of a candidate lymphoma drug comprising:
a) administering said drug to a patient;
b) removing a cell sample from said patient; and
c) determining alterations in the expression or activation of a gene comprising a nucleic acid sequence selected from the group consisting of the nucleic acid sequences set forth in SEQ ID NO:1 and SEQ ID NO:3.

11. A method of diagnosing lymphoma comprising:
a) determining the expression of one or more genes selected from the group consisting of the nucleic acid sequences set forth in SEQ ID NO:1 and SEQ ID NO:3,. or a polypeptide encoded thereby, in a first tissue type of a first individual; and
b) comparing said expression of said gene(s) from a second normal tissue type from said first individual or a second unaffected individual;
wherein a difference in said expression indicates that the first individual has lymphoma.

12. A method for inhibiting the activity of an Nrf2 protein, wherein said Nrf2 protein comprises an amino acid sequence encoded by a nucleic acid sequence selected from the group consisting of the nucleic acid sequences set forth in SEQ ID NO:1 and SEQ ID NO:3, said method comprising binding an inhibitor to said Nrf2 protein.

13. A method of treating lymphoma comprising administering to a patient an inhibitor of an Nrf2 protein, wherein said Nrf2 protein comprises an amino acid sequence encoded by a nucleic acid sequence selected from the group consisting of the nucleic acid sequences set forth in SEQ ID NO:1 and SEQ ID NO:3.

14. A method of neutralizing the effect of an Nrf2 protein, wherein said Nrf2 protein comprises an amino acid sequence encoded by a nucleic acid sequence selected from the group consisting of the nucleic acid sequences set forth in SEQ ID NO:1 and SEQ ID NO:3, comprising contacting an agent specific for said Nrf2 protein with said Nrf2 protein in an amount sufficient to effect neutralization.

15. A polypeptide which specifically binds to a protein comprising an amino acid sequence encoded by a nucleic acid sequence selected from the group consisting of the nucleic acid sequences set forth in SEQ ID NO:1, SEQ ID NO:3, Genbank accession number U20532, and Genbank accession number NM_{—}006164.

16. A polypeptide according to claim 15, which comprises an antibody.

17. A biochip comprising one or more nucleic acid fragments from a nucleic acid selected from the group consisting of the nucleic acid sequences set forth in SEQ ID NO:1, SEQ ID NO:3, and complements thereof.

18. A method of diagnosing lymphomas or a propensity to lymphomas by sequencing at least one Nrf2 gene of an individual.

19. A method of determining Nrf2 gene copy number comprising adding an Nrf2 gene probe to a sample of genomic DNA from an individual under conditions suitable for hybridization.
